(19) United States
(12) Patent Application Publication
(10) Pub. No.: US 2024/0103002 A1
(43) Pub. Date: Mar. 28, 2024

(54) ORTHOPOXVIRUS SEROLOGY ASSAYS

(71) Applicant: Meso Scale Technologies, LLC., Rockville, MD (US)

(72) Inventors: George SIGAL, Rockville, MD (US); Leonid DZANTIEV, Ellicott City, MD (US); Priscilla KRAI, Germantown, MD (US); Teri-Tu B. NGO, Ashburn, VA (US)

(21) Appl. No.: 18/473,064

(22) Filed: Sep. 22, 2023

Related U.S. Application Data
(60) Provisional application No. 63/513,390, filed on Jul. 13, 2023.
(60) Provisional application No. 63/376,828, filed on Sep. 23, 2022.

(51) Int. Cl. G01N 33/569

(52) U.S. Cl. CPC G01N 33/56983; G01N 2333/065; G01N 2333/07; G01N 2469/20

(57) ABSTRACT

The invention relates to methods and kits for detecting one or more antibody biomarkers in a sample, wherein the antibody biomarker specifically binds a viral antigen. In embodiments, the viral antigen is a monkeypox virus (MPXV) antigen, a vaccinia virus (VACV) antigen, or combination thereof.

### FIELD OF THE INVENTION

The invention relates to methods and kits for detecting biomarker(s), e.g., antibody biomarkers that specifically bind a viral antigen. In embodiments, the viral antigen is an orthopoxvirus antigen. In embodiments, the orthopoxvirus is monkeypox virus (MPXV) or vaccinia virus (VACV).

### BACKGROUND

Orthopoxviruses, including smallpox, monkeypox, and vaccina viruses, can cause a number of contagious infections and can be fatal. The 2022 monkeypox outbreak highlights the need for assays for multiple reasons, e.g., to detect infection, to aid in the development of vaccines, to follow the immune status and past viral exposure of individuals, to identify individuals who are immune or at low risk of infection, and for epidemiological studies.

### SUMMARY OF THE INVENTION

In embodiments, the invention provides a kit for detecting one or more antibody biomarkers of interest in a sample, the kit comprising, in one or more vials, containers, or compartments: (a) a surface comprising a panel of antigens immobilized on one or more binding domains, wherein each binding domain comprises an antigen of the panel of antigens immobilized thereon, wherein the panel of antigens comprises a monkeypox virus (MPXV) protein, a vaccinia virus (VACV) protein, or combination thereof; and (b) one or more detection reagents, wherein each detection reagent comprises a detection antibody, a detection antigen, or a detection competitor.

In embodiments, the invention provides a method of detecting one or more antibody biomarkers of interest in a sample, comprising: (a) contacting the sample with a surface comprising a panel of antigens immobilized on one or more binding domains, wherein each binding domain comprises an antigen of the panel of antigens immobilized thereon, wherein the panel of antigens comprises a monkeypox virus (MPXV) protein, a vaccinia virus (VACV) protein, or combination thereof; (b) forming a binding complex in each binding domain, wherein each binding complex comprises the antigen and an antibody biomarker that binds to the antigen; (c) contacting the binding complex in each binding domain with a detection reagent; and (d) detecting the binding complexes on the surface, thereby detecting the one or more antibody biomarkers in the sample.

In embodiments, the invention provides a method of detecting one or more antibody biomarkers of interest in a sample, comprising: (a) forming one or more binding complexes, wherein each binding complex comprises an antigen; an antibody biomarker that binds to the antigen; and a detection reagent, wherein the antigen comprises an MPXV protein, a VACV protein, or combination thereof; and (b) detecting the one or more binding complexes, thereby detecting the one or more antibody biomarkers in the sample.

### BRIEF DESCRIPTION OF THE DRAWINGS

FIG. 1 is adapted from NCBI Insights, May 26, 2022: “Monkeypox virus: Complete genome from the current outbreak now available in GenBank,” and shows an exemplary phylogenomic tree of monkeypox virus genomes. The 2022 monkeypox outbreak isolate, designated as GenBank accession no. ON563414, is highlighted in grey.

FIGS. 2A and 2B illustrate exemplary assay surfaces described in embodiments herein. FIG. 2A shows a well of an exemplary 384-well assay plate, comprising four distinct binding domains (“spots”). FIG. 2B shows a well of an exemplary 96-well assay plate, comprising ten distinct binding domains (“spots”).

### DETAILED DESCRIPTION OF THE INVENTION

In embodiments, the invention aids in assessing human immune responses to orthopoxvirus (e.g., monkeypox virus and/or vaccinia virus) infection and vaccination. The serology assays provided herein are conducted in a simple and streamlined format with improved sensitivity.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” (whether italicized or not) means that the specific terms recited are representative examples and embodiments of the invention that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

As used herein, “host” refers to a subject who has been infected with or suspected of being infected with a virus described herein, e.g., an orthopoxvirus such as monkeypox virus. In embodiments, the host is a human subject.

As used herein, a “nucleic acid” refers to one (i.e., a single nucleotide monomer) or more nucleotides. In embodiments where “nucleic acid” refers to more than one nucleotides, the nucleotides may be covalently linked to form a polymeric structure, e.g., a “polynucleotide,” “oligonucleotide,” or “nucleic acid sequence.”

As used herein, a “polypeptide” refers to a substance composed of amino acids linearly linked by amide bonds (i.e., peptide bonds). The term “polypeptide” refers to any chain or chains of amino acids, and does not refer to a specific length of the product. Thus, peptides, dipeptides, tripeptides, oligopeptides, protein, amino acid chain, or any other term used to refer to a chain or chains of amino acids, are included within the definition of “polypeptide,” and the term “polypeptide” may be used instead of or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The terms “antibody,” “antibody biomarker,” and “immunoglobulin” are used interchangeably herein. An antibody comprises at least the variable domain of a heavy chain, and typically comprises at least the variable domains of a heavy chain and a light chain. In embodiments, antibodies of the present invention bind to one or more proteins of an orthopoxvirus described herein, e.g., monkeypox virus and/or vaccinia virus.

As used herein, the term “antigen-binding substance” refers to antibodies, antibody fragments, antibody derivatives, antibody analogues, antibody variants, engineered antibodies, and other substances that bind to antigens in a manner similar to antibodies. Antigen-binding substances include substances that comprise at least one heavy or light chain complementarity determining region (CDR) of an antibody. In embodiments, antigen-binding substances of the present invention bind to one or more proteins of an orthopoxvirus described herein, e.g., monkeypox virus and/or vaccinia virus.

As used herein, “antigen” refers to a substance that is capable of specifically or preferentially binding an antibody-binding substance such as an antibody or antigen-binding fragment thereof. In embodiments, antigens of the present invention comprise an orthopoxvirus protein described herein, e.g., monkeypox virus and/or vaccinia virus.

As used herein, the term “biomarker” refers to a biological substance that is indicative of a normal or abnormal process, e.g., disease, infection, or environmental exposure. Biomarkers can be small molecules such as ligands, signaling molecules, or peptides, or macromolecules such as antibodies, receptors, or proteins and protein complexes. A change in the levels of a biomarker can correlate with the risk or progression of a disease or abnormality or with the susceptibility or responsiveness of the disease or abnormality to a given treatment. A biomarker can be useful in the diagnosis of disease risk or the presence of disease in an individual, or to tailor treatments for the disease in an individual (e.g., choices of drug treatment or administration regimes). In evaluating potential drug therapies, a biomarker can be used as a surrogate for a natural endpoint such as survival or irreversible morbidity. If a treatment alters a biomarker that has a direct connection to improved health, the biomarker serves as a “surrogate endpoint” for evaluating clinical benefit. Biomarkers are further described in, e.g., Mayeux, NeuroRx 1(2): 182-188 (2004); Strimbu et al., Curr Opin HIV AIDS 5(6): 463-466 (2010); and Bansal et al., Statist Med 32: 1877-1892 (2013). The term “biomarker,” when used in the context of a specific organism (e.g., human, nonhuman primate or another animal), refers to the biomarker native to that specific organism. Unless specified otherwise, the biomarkers referred to herein encompass human biomarkers.

As used herein, the term “level” in the context of a biomarker refers to the amount, concentration, or activity of a biomarker. The term “level” can also refer to the rate of change of the amount, concentration, or activity of a biomarker. A level can be represented, for example, by the amount or synthesis rate of messenger RNA (mRNA) encoded by a gene, the amount or synthesis rate of polypeptide corresponding to a given amino acid sequence encoded by a gene, or the amount or synthesis rate of a biochemical form of a biomarker accumulated in a cell, including, for example, the amount of particular post-synthetic modifications of a biomarker such as a polypeptide (e.g., an antibody), nucleic acid, or small molecule. “Level” can also refer to an absolute amount of a biomarker in a sample or to a relative amount of the biomarker, including amount or concentration determined under steady-state or non-steady-state conditions. “Level” can further refer to an assay signal that correlates with the amount, concentration, activity or rate of change of a biomarker. The level of a biomarker can be determined relative to a control marker in a sample.

A “serology assay” is an assay used to identify antigen-binding substances, e.g., antibodies or fragments thereof, in a sample, which may include a body or non-bodily fluid as further described herein. In embodiments, the serology assays provided herein detect antigen-binding substances that specifically bind orthopoxvirus antigens, e.g., MPXV and/or VACV antigens.

A “panel,” as used in the context of the assays and kits of the invention, is a combination of biological substances, e.g., antigens and/or biomarkers described herein, that may be measured in a multiplexed format. In embodiments, a “panel of antigens” refers to a plurality of antigens.

### Orthopoxviruses

Orthopoxviruses, which belong to the Poxviridae family of viruses, are enveloped viruses with brick-shaped geometries and a linear DNA genome. Currently known species in the Orthopoxvirus genus of viruses include abatino macacapox virus, akhmeta virus, Alaskapox virus, camelpox virus, cowpox virus, ectromelia virus, monkeypox virus (MPXV), raccoonpox virus, skunkpox virus, taterapox virus, vaccinia virus (VACV), variola virus (VARV; also known as smallpox virus), and volepox virus. Human-specific smallpox has been eradicated, while monkeypox, cowpox, and vaccinia virus infections occur throughout the world, generally via zoonotic transmission. Infection with orthopoxviruses, e.g., MPXV, can lead to localized or generalized skin lesions, progressing from papules to vesicles and scabs. Other symptoms include, e.g., fever, chills, swollen lymph nodes, exhaustion, muscle aches, backache, headache, and respiratory symptoms (e.g., sore throat, nasal congestion, or cough).

In general, two distinct infectious virus particles exist: an intracellular mature virus (IMV) and extracellular enveloped virus (EEV). Intracellular and cell-associated viruses are involved in the spreading of the virus from cell to cell, whereas viruses released from the cell enable the dissemination within the infected organism. See, e.g., Pauli et al., Transfus Med Hemother 37(6):351-364 (2010).

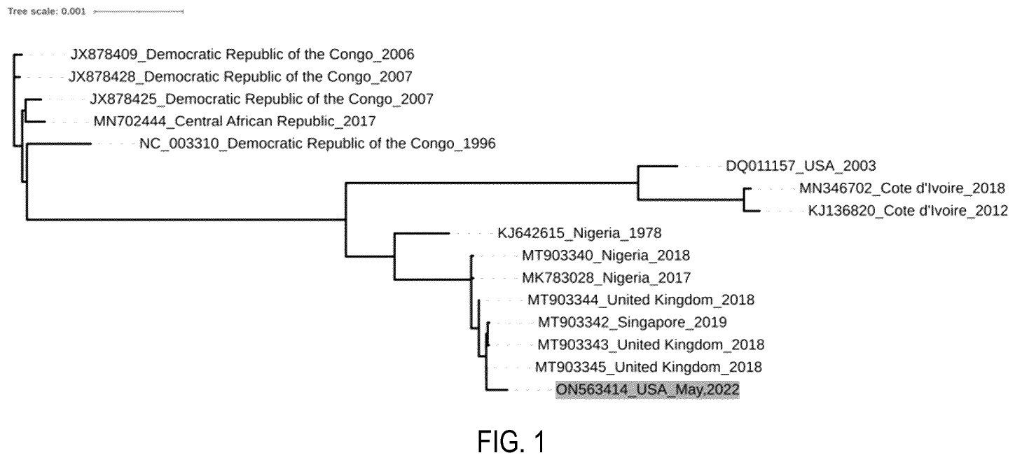

Genome sequences of the orthopoxviruses described herein, e.g., MPXV and VACV, can be accessed through the NCBI Virus Database. Genetically, monkeypox viruses cluster into two groups: the Congo basin and the west African clade. A representative phylogenomic tree of MPXV genomes is shown in FIG. 1. Unless otherwise specified, the MPXV antigens described herein are based on NCBI GenBank accession no. ON563414, highlighted in grey in FIG. 1, i.e., the isolate of the 2022 monkeypox outbreak. GenBank accession no. ON563414 is also known as the “MPXV_USA_2022_MA001” isolate. See, e.g., NCBI Insights, May 26, 2022: “Monkeypox virus: Complete genome from the current outbreak now available in GenBank”; available at: www.ncbiinsights.ncbi.nlm.nih.gov/2022/05/26/monkeypox-virus-genome. MPXV antigens of interest include, e.g., E8L, which may be involved in attachment to a host cell by binding cell surface chondroitin sulfate; and A30L, which may be involved in cell membrane fusion and syncytial formation.

Various vaccinia virus strains include, e.g., the non-replicative Modified Vaccinia Ankara (MVA) strain, the attenuated Lister strain, the attenuated LC16m8 strain, the virulent WR strain, and the Connaught strain developed by Connaught Laboratories. Further VACV strains are described in, e.g., Qin et al., J Virol 85(24):13049-13060 (2011) and de Freitas et al., J Virol 93(6):e02191-18 (2019). Cross-reactive immune responses between MPXV and VACV have been observed, i.e., vaccination with one or more antigens from one virus elicits a neutralizing antibody response against the other virus. See, e.g., Gao et al., Virology Journal 20:126 (2023); Manenti et al., Frontiers in Public Health 11:1195674 (2023); Tang et al., Frontiers in Immunology 14:1203410 (2023); Zaeck et al., Nature Medicine 29:270-278 (2023).

VACV has been used in vaccines against orthopoxviruses, including those that eradicated smallpox. Monkeypox virus (MPXV) vaccines comprise VACV antigens from smallpox. Therefore, no MPXV vaccine comprises antigens specific only to MPXV. For example, the JYNNEOS vaccine is based on the MVA strain. Further, a live-orthopoxvirus vaccine has been developed based on antigens from VACV strain Connaught, termed the “4pox” vaccine. See, e.g., Hooper et al., Virology 306(1):181-195 (2003); Hooper et al., J Virol 78(9):4433-4443 (2004); and Golden et al., Clin Vaccine Immunol 17(11):1656-1665 (2010). The 4pox vaccine includes four VACV structural proteins: L1R, A27L, A33R, and B5R. The L1R protein participates in virion assembly and has a role in viral entry and maturation; the A27L protein promotes the fusion of viral and host plasma membranes; the A33R protein may affect intercellular diffusion of virions; and the B5R protein is a known target of neutralizing antibodies. Further viral proteins that are shown or expected to elicit an immune response include, e.g., the VACV proteins H3L, A14L, D8L, B8R, B18R, B19R, A17L, and A28L. See, e.g., Singh et al., J Virol 90:5020-4030 (2016); Meng et al., Virology 418:284-292 (2018); Borovkov et al., Virology 395:97-113 (2009); Ahmed et al., bioRxiv doi.org/10.1101/2022.06.23.497143 (2022); Cohn et al., medRxiv doi.org/10.1101/2023.03.07.23286701 (2023). Unless otherwise specified, the VACV antigens described herein are based on the Lister strain with NCBI GenBank accession no. DQ121394.1, also known as “VACV strain Lister clone VACV107.”

### Immunoassay Methods

In embodiments, the invention provides a method for detecting a biomarker that is produced by a host (e.g., a human subject) in response to a viral infection, e.g., by an orthopoxvirus. In embodiments, the orthopoxvirus is monkeypox virus (MPXV). In embodiments, the orthopoxvirus is vaccinia virus (VACV). Unless otherwise specified, the biomarkers described herein are produced by a host, e.g., a human subject, in response to viral exposure and/or infection as described herein. In embodiments, the biomarker is an immune response biomarker. In embodiments, the biomarker is an antibody. The terms “antibody biomarker” and “antibody” are used interchangeably throughout the present disclosure. In embodiments, the biomarker is a neutralizing antibody. In embodiments, a neutralizing antibody is an antibody that defends a host from a pathogen by influencing how molecules on the pathogen's surface can enter a host cell. In embodiments, a neutralizing antibody is capable of preventing a pathogen from altering its structure and shape to enter and replicate within a host cell. In embodiments, the method is used to assess the severity and/or prognosis of a viral infection in a subject. In embodiments, the method is used to determine whether a subject has been previously exposed to a virus. In embodiments, the method is used to estimate the time of virus exposure and/or infection. In embodiments, the method is used to determine whether a subject has immunity to a virus. In embodiments, the method is used to determine the vaccination status of a subject.

Measurement of biomarker values and levels before and after a particular event, e.g., cellular or environmental event, may be used to gain information regarding an individual's response to the event. For example, samples or model organisms can be subjected to stress- or disease-inducing conditions, or a treatment or prevention regimen, and a particular biomarker can then be detected and quantitated in order to determine its changes in response to the condition or regimen. However, the opposite, i.e., measuring biomarker values and levels to determine whether an organism has been subjected to stress- or disease-inducing condition, tends to be much more complicated, as changes in the levels of a single biomarker are sometimes not definitively associated with a particular condition.

In embodiments, the measured levels of the one or more biomarkers described herein provides information regarding infection and immune response to infection, e.g., the course or maturity of infection, the etiology of severe illness, and the potential severity of illness. In embodiments, the measured levels of the one or more biomarkers described herein provides information regarding a subject's antibody response, cytokine response, neutrophil, macrophage, and/or monocyte production, complement activation, B cell and/or T cell activation, or a combination thereof.

In embodiments, detection and/or measurement of a single biomarker is sufficient to provide a prediction and/or diagnosis of a disease or condition. In embodiments, combinations of biomarkers are used to provide a strong prediction and/or diagnosis. Although a linear combination of biomarkers (i.e., the combination comprises biomarkers that individually provide a relatively strong correlation) can be utilized, linear combinations may not be available in many situations, for example, when there are not enough biomarkers available and/or with strong correlation. In alternative approaches, a biomarker combination is selected such that the combination is capable of achieving improved performance (i.e., prediction or diagnosis) compared with any of the individual biomarkers, each of which may not be a strong correlator on its own. Biomarkers for inclusion in a biomarker combination can be selected for based on their performance in different individuals, e.g., patients, wherein the same biomarker may not have the same performance in different individuals, but when combined with the remaining biomarkers, provide an unexpectedly strong correlation for prediction or diagnosis in a population. For example, Bansal et al., Statist Med 32: 1877-1892 (2013) describe methods of determining biomarkers to include in such a combination, noting in particular that optimal combinations may not be obvious to one of skill in the art, especially when subgroups are present or when individual biomarker correlations are different between cases and controls. Thus, selecting a combination of biomarkers for providing a consistent and accurate prediction and/or diagnosis can be particularly challenging and unpredictable.

Even when a suitable combination of biomarkers is determined, utilizing the combination of biomarkers in an assay poses its own set of difficulties. For example, detecting and/or quantitating each biomarker in the combination in its own separate assay may not be feasible with small samples, and using a separate assay to measure each biomarker in a sample may not provide consistent and comparable results. Furthermore, running an individual assay for each biomarker in a combination can be a cumbersome and complex process that can be inefficient and costly.

In embodiments, the method described herein is an immunoassay, e.g., sandwich immunoassay, which comprises using a binding reagent and a detection reagent that each specifically bind to an analyte, e.g., a biomarker of interest as described herein. Unless otherwise specified, the binding reagent of the immunoassays provided herein comprises a viral antigen described herein, e.g., an MPXV and/or VACV antigen. Binding and detection reagents are further described herein. In embodiments, the immunoassay described herein is a multiplexed immunoassay method. A multiplexed assay that can simultaneously measure the concentrations of multiple biomarkers can provide reliable results while reducing processing time and cost. Challenges of developing a multi-biomarker assay (such as, e.g., a multiplexed assay described in embodiments herein) include, for example, determining compatible reagents for all of the biomarkers (e.g., capture and detection reagents described herein should be highly specific and not be cross-reactive; all assays should perform well in the same diluents); determining concentration ranges of the reagents for consistent assay (e.g., comparable capture and detection efficiency for the assays described herein); having similar levels in the condition and sample type of choice such that the levels of all of the biomarkers fall within the dynamic range of the assays at the same dilution; minimizing non-specific binding between the biomarkers and binding reagents thereof or other interferents; and accurately and precisely detecting a multiplexed output measurement.

In embodiments, the method provided herein is used to diagnose whether a subject is infected with a virus. In embodiments, the method is used to assess the severity and/or prognosis of a viral infection in a subject. In embodiments, the method is used to determine whether a subject has been previously exposed to a virus. In embodiments, the method is used to estimate the time of virus exposure and/or infection. In embodiments, the method is used to determine whether a subject has immunity to a virus. In embodiments, the virus is MPXV.

In embodiments, the invention provides methods of assessing an individual's immune response to a viral infection. In embodiments, the invention provides methods of assessing a group of individuals immune response to a viral infection. In embodiments, assessing an immune response comprises determining the type and/or strength of the immune response, e.g., detecting the molecular components produced in response to a viral infection (e.g., acute phase reactants, antibodies, cytokines, etc.) and measuring the amounts of each component produced. In embodiments, the invention provides methods of assessing the differences in immune responses by age, race, ethnicity, socioeconomic backgrounds, and/or comorbidities and underlying conditions, e.g., HIV/AIDS infection, leukemia, lymphoma, autoimmune conditions, atopic dermatitis, and other active exfoliative skin conditions, which may be associated with severe illness and/or poor clinical outcomes from the viral infection. In embodiments, the invention provides methods of determining the epidemiology of diseases caused by the viruses described herein, e.g., monkeypox. In embodiments, the virus is an orthopoxvirus, e.g., MPXV.

In embodiments, the method provided herein is used to identify individuals with previous virus exposure for epidemiological studies (e.g., to understand true disease prevalence and evaluate the efficacy of infection control measures). In embodiments, the method is used to identify individuals at higher or lower risk of future infection. Moreover, the method can be an important tool in the research, development, and validation of a vaccine for the virus.

In embodiments, the method is used to assess differences in immune responses (e.g., antibody response) between individuals whose immunity is achieved by natural infection or vaccination. For example, a multiplexed method differentiates an individual's response to vaccination with an antigen from one orthopoxvirus, e.g., VACV, compared with the individual's response to natural infection by a different orthopoxvirus, e.g., MPXV. Such a method can advantageously distinguish between individuals with biomarkers produced in response an active infection and are potentially contagious and individuals with biomarkers produced in response to the vaccine. In embodiments, the virus is an orthopoxvirus, e.g., MPXV. In embodiments, an individual who has recovered from an infection by MPXV (“infected recoveree”) has a stronger immune response as compared to an individual vaccinated with an antigen from VACV (“vaccinee”). In embodiments, an infected recoveree has higher levels of neutralizing antibodies against an MPXV and/or VACV antigen as compared with a vaccinee. See, e.g., Cohn et al., medRxiv doi.org/10.1101/2023.03.07.23286701 (2023) and Yefet et al., iScience 26: 105957 (2023).

In embodiments, the invention provides methods of assessing cross-reactivity of an individual's immune response between different orthopoxviruses (e.g., MPXV and VACV). In embodiments, the invention provides methods of mapping the epitopes recognized by an individual's immune response, e.g., epitopes on a viral protein from MPXV or VACV. In embodiments, the invention provides methods of assessing the individual's clinical outcome based on the mapped epitopes of immune responses. In embodiments, the invention provides methods of assessing an individual's immune response by detecting different IgG classes and/or subclasses. In embodiments, the invention provides methods of assessing the individual's clinical outcome based on the IgG classes and/or subclasses. In embodiments, the invention provides methods of assessing the affinity and/or avidity of an individual's immune response to different viral antigens. In embodiments, the invention provides methods of assessing the strength of an immune response, e.g., measuring the total antibody concentration or the concentration of different classes or subclasses of antibodies in an individual. In embodiments, the invention provides methods of determining the natural interacting partner(s) of the virus, e.g., an orthopoxvirus such as MPXV and/or VACV. As used in the context of viral infections, a “natural interacting partner” refers to a substance in the host cell (e.g., proteins or carbohydrate moieties on a host cell surface) that interacts with a viral component described herein. Natural interacting partners of viruses are further described in, e.g., Brito et al., Front Microbiol 8:1557 (2017).

In embodiments, the invention provides methods of assessing changes in the immune response over time. In embodiments, the invention provides methods of assessing an individual's immune response at different time points after infection and/or after the first onset of a symptom. In embodiments, the invention provides methods of assessing immune response components (e.g., antibodies) present in an individual at different time points after infection and/or after the first onset of a symptom. Symptoms of viral infections are described herein. In embodiments, the invention provides methods of assessing the long-term effects of an infection on an individual. In embodiments, the invention provides methods of assessing an individual's immune response at different time points after vaccination. In embodiments, the invention provides methods of determining the immune response components (e.g., antibodies) that provide immunity to a viral infection. In embodiments, the invention provides methods of assessing an individual's immune response at different time points after receiving a treatment for the viral infection. In embodiments, the invention provides methods of assessing the effect of convalescent serum treatment in an individual, e.g., comprising measuring the individual's immune response after administration of the convalescent serum. In embodiments, the invention provides methods of assessing the immune response components (e.g., antibodies) present in a convalescent serum sample, e.g., comprising determining its effectiveness, half-life, and/or functional window of treatment in an individual. In embodiments, the invention provides methods of assessing the effectiveness, half-life, and/or functional window of protection of a therapeutic antibody treatment. In embodiments, the virus is an orthopoxvirus, e.g., MPXV. In embodiments, the invention provides methods of assessing an individual's immune response, e.g., an antibody, to vaccination against one orthopoxvirus, e.g., VACV, to determine a clinical outcome of infection by a different orthopoxvirus, e.g., MPXV.

In embodiments, the invention provides a serology assay for determining the MPXV strain that has infected an individual. In embodiments, the invention provides a method for tracking spread of one or more MPXV strains. In embodiments, the invention provides a method for tracking the spread of one or more MPXV strains in one or more geographical regions and/or for tracking the spread of one or more MPXV strains over time.

In some embodiments, the sample is from one or more individuals, wherein the one or more individuals are currently infected with MPXV. In some embodiments, the sample is from one or more individuals, wherein the one or more individuals were previously infected with MPXV. In some embodiments, the sample is from at least two individuals, wherein at least one individual is currently infected with MPXV and at least one individual was previously infected with MPXV. In some embodiments, the sample is from at least one individual, wherein the individual is currently infected and was previously infected with MPXV. In embodiments, the sample is from one or more individuals, wherein the one or more individuals are located in one or more geographical regions, thereby tracking spread of the MPXV in the one or more geographical regions. In embodiments, the sample is from one or more individuals obtained at different time points. In embodiments, the sample comprises a pooled sample from at least two individuals. Pooled samples are further described herein.

In embodiments, the invention provides improved sensitivity and/or specificity in determining whether a subject is currently infected or has previously been infected with a virus, e.g., an orthopoxvirus such as MPXV. In embodiments, the invention provides improved sensitivity and/or specificity in determining whether a subject has immunity to a virus, e.g., an orthopoxvirus such as MPXV. In embodiments, the invention provides improved sensitivity and/or specificity in determining whether a subject has been vaccinated, e.g., with a VACV antigen, against infection by a different orthopoxvirus, e.g., MPXV. In embodiments, the methods herein have a sensitivity of greater than 90%, greater than 95%, greater than 96%, greater than 97%, greater than 98%, greater than 99%, greater than 99.5%, or greater than 99.9%. In embodiments, the methods herein have a specificity of greater than 90%, greater than 95%, greater than 96%, greater than 97%, greater than 98%, greater than 99%, greater than 99.5%, or greater than 99.9%. Assays with high sensitivity and specificity are important to correctly diagnose active infections and to correctly determine whether an individual has been previously exposed and/or immune to a virus, e.g., an orthopoxvirus such as MPXV. In particular, assays with high specificity are useful for conducting epidemiological studies in populations with low disease prevalence. Moreover, assays with high specificity are important for individual assessment due to the high risk of a false positive to the individual and the individual's community; individuals who received a false positive serology test result for MPXV and/or VACV may believe themselves to be immune and therefore erroneously engage in activity that can increase the likelihood of infection and spread of the virus.

In embodiments, the invention provides a method for detecting a biomarker that is capable of binding to a viral antigen in a sample. As used herein, a virus or viral antigen is any component or secretion of a virus that prompts an immune response in a host (e.g., a human). In embodiments, the viral antigen is a viral protein or fragment thereof. In embodiments, the method is capable of determining whether a subject has been exposed to a particular virus, e.g., an orthopoxvirus such as MPXV, and/or vaccinated against orthopoxvirus infection, e.g., with a VACV antigen. In embodiments, the method is capable of determining whether a subject is at risk of being infected by a particular virus, e.g., an orthopoxvirus such as MPXV. In embodiments, the method is capable of determining whether a subject has immunity to a particular virus, e.g., an orthopoxvirus such as MPXV.

In embodiments, the biomarker is a human biomarker. In embodiments, the biomarker is a nonhuman primate (NHP) biomarker. NHPs include, e.g., macaques such as rhesus monkeys, cynomolgus monkeys, and pig-tailed monkeys; vervet monkeys; baboons; squirrel monkeys; and owl monkeys. In embodiments, the biomarker is a mouse biomarker or a rat biomarker. In embodiments, the biomarker is an IgA or antigen-binding fragment thereof capable of binding to an MPXV and/or VACV antigen described herein. In embodiments, the biomarker is an IgG or antigen-binding fragment thereof capable of binding to an MPXV and/or VACV antigen described herein. In embodiments, the biomarker is an IgG1 or antigen-binding fragment thereof capable of binding to an MPXV and/or VACV antigen described herein. In embodiments, the biomarker is an IgG2 or antigen-binding fragment thereof capable of binding to an MPXV and/or VACV antigen described herein. In embodiments, the biomarker is an IgG3 or antigen-binding fragment thereof capable of binding to an MPXV and/or VACV antigen described herein. In embodiments, the biomarker is an IgG4 or antigen-binding fragment thereof capable of binding to an MPXV and/or VACV antigen described herein. In embodiments, the biomarker is an IgM or antigen-binding fragment thereof capable of binding to an MPXV and/or VACV antigen described herein. In embodiments, the biomarker is an IgE or antigen-binding fragment thereof capable of binding to an MPXV and/or VACV antigen described herein. In embodiments, the biomarker is an IgD or antigen-binding fragment thereof capable of binding to an MPXV and/or VACV antigen described herein.

In embodiments, the biomarker capable of binding to a viral antigen is an immune biomarker. In embodiments, the biomarker is an antibody or antigen-binding fragment thereof. In embodiments, the biomarker is an immunoglobulin A (IgA), immunoglobulin G (IgG; including IgG subclasses IgG1, IgG2, IgG3, and IgG4), immunoglobulin M (IgM), immunoglobulin E (IgE), or immunoglobulin D (IgD), or antigen-binding fragments thereof capable of binding to the viral antigens described herein, e.g., MPXV and/or VACV antigens. IgA, IgG (and subclasses thereof), IgM, IgE, and IgD are different isotypes of antibodies that have different immunological properties and functional locations. For example, IgA is typically found in the mucosal areas, such as the respiratory and gastrointestinal tracts, saliva, and tears and can prevent colonization by pathogens. IgG, the most abundant antibody isotype, has four subclasses as described herein and is found in all bodily fluids and provides the majority of antibody-based immunity against pathogens. IgM is mainly found in the blood and lymph fluid and is typically the first antibody made by the body to fight a new infection. IgE is mainly associated with allergic reactions (e.g., as part of aberrant immune response) and is found in the lungs, skin, and mucous membranes. IgD mainly functions as an antigen receptor on B cells and may activate basophils and mast cells to produce antimicrobial factors. Based on the timing and/or type of infection, different amounts of each isotype are produced.

In embodiments, the method is a multiplexed immunoassay method capable of quantifying the amount of each isotype of antibodies, e.g., IgG, IgA, IgE, and IgM, present in the sample. In embodiments, the amounts of the different isotypes of antibodies measured in a sample, e.g., the amounts of each of IgG, IgA, IgE, and IgM, can be used to determine whether a subject has been previously exposed to a virus. In embodiments, the amounts of the different isotypes of antibodies measured in a sample, e.g., the amounts of each of IgG, IgA, IgE, and IgM, can be used to estimate the time of virus exposure and/or infection. In embodiments, the amounts of the different isotypes of antibodies measured in a sample, e.g., the amounts of each of IgG, IgA, IgE, and IgM, can be used to determine whether a subject has immunity to a virus, e.g., an orthopoxvirus such as MPXV.

IgG is further divided into four subclasses, IgG1, IgG2, IgG3, and IgG4, based on properties such as ability to activate complement, bind to macrophages, and/or pass through the placenta. Each subclass also has a distinct biological function. For example, the response to protein antigens is primarily mediated by IgG1 and IgG3, while IgG2 primarily mediates the response to polysaccharide antigens. IgG4 plays a role in protection against certain hypersensitivity reactions and pathogenesis of some autoimmune diseases. IgG subclass screening is performed to monitor a subject's infection response and/or determine whether a subject has antibody deficiency, and/or assess a subject's risk of an adverse response to infection.

In embodiments, the method of detecting an antibody biomarker, e.g., IgG, IgA, IgE, and/or IgM, in a sample comprises: (a) forming at least a first, second, third, and fourth binding complex comprising (i) a first, second, third, and fourth viral antigens, respectively, wherein each viral antigen specifically binds to IgG, IgA, IgE, and IgM, respectively, and (ii) IgG, IgA, IgE, or IgM; and (b) measuring the concentration of IgG, IgA, IgE, or IgM in each of the binding complexes. In embodiments, each of the first, second, third, and fourth viral antigens is an MPXV and/or a VACV antigen as described herein. In embodiments, the IgG, IgA, IgE, and/or IgM is from a human, NHP, mouse, rat, combination thereof.

In embodiments, the method of detecting an antibody biomarker, e.g., IgG1, IgG2, IgG3, and/or IgG4, in a sample comprises: (a) forming at least a first, second, third, and fourth binding complex comprising (i) a first, second, third, and fourth viral antigens, respectively, wherein each viral antigen specifically binds to IgG1, IgG2, IgG3, and IgG4, respectively, and (ii) IgG1, IgG2, IgG3, or IgG4; and (b) measuring the concentration of IgG1, IgG2, IgG3, or IgG4 in each of the binding complexes. In embodiments, each of the first, second, third, and fourth viral antigens is an MPXV and/or a VACV antigen as described herein. In embodiments, the IgG, IgA, IgE, and/or IgM is from a human, NHP, mouse, rat, combination thereof.

In embodiments, the method of detecting an antibody biomarker, e.g., IgG1, IgG2, IgG3, IgG4, IgA, IgE, and/or IgM, in a sample comprises: (a) forming a plurality of binding complexes, each comprising a viral antigen, wherein the viral antigen specifically binds to an antibody selected from IgG1, IgG2, IgG3, IgG4, IgA, IgE, and IgM; and the antibody; and (b) measuring the concentration of the antibody in each of the binding complexes. In embodiments, each of the first, second, third, and fourth viral antigens is an MPXV and/or a VACV antigen as described herein. In embodiments, the IgG1, IgG2, IgG3, IgG4, IgA, IgE, and/or IgM is from a human, NHP, mouse, rat, combination thereof.

In embodiments, the invention provides a method of detecting one or more antibody biomarkers of interest in a sample, comprising: (a) forming one or more binding complexes, wherein each binding complex comprises an antigen; an antibody biomarker that binds to the antigen; and a detection reagent; and (b) detecting the one or more binding complexes, thereby detecting the one or more antibody biomarkers in the sample. In embodiments, the antigen comprises an MPXV protein, a VACV protein, or combination thereof. In embodiments, the biomarker is IgG, IgA, IgM, or combination thereof. In embodiments, the method is an immunoassay method, e.g., a classical, bridging, or competitive serology assay described herein.

In embodiments, the invention provides an immunoassay method comprising quantifying the amounts of one or more biomarkers capable of binding to an MPXV antigen and/or a VACV antigen in a sample. In embodiments, the immunoassay method comprises: (a) forming a binding complex comprising a viral antigen and a biomarker from the sample that binds to the viral antigen; and (b) measuring the concentration of the biomarker in the binding complex. In embodiments, the biomarker is IgG, IgA, IgM, or combination thereof. In embodiments, the biomarker is human IgG, IgA, or IgM. In embodiments, the biomarker is mouse IgG, IgA, or IgM. In embodiments, the biomarker is rat IgG, IgA, or IgM. In embodiments, the binding complex further comprises a detection reagent that specifically binds IgG, IgA, or IgM, and the concentration of the biomarker is measured by detecting the detection reagent. Detection reagents are further described herein. In embodiments, the immunoassay method is a classical serology assay. In embodiments, the immunoassay method is a bridging serology assay. In embodiments, the immunoassay method is a competitive serology assay. Classical, bridging, and competitive serology assays are further described herein.

In embodiments, the immunoassay detects an antibody biomarker that binds to any of the antigens as shown in Table 1.

TABLE 1
MPXV and VACV Antigens

| Group | Antigens |
|---|---|
| 1 | L1R, A27L, A33R, B5R, H3L, A14L, D8L, |
| | B8R, B19R, A17L, A28L, and/or B18R |
| | proteins from VACV, and/or an |
| | MPXV homolog thereof |
| 2 | M1R, A29L, A35R, B6R, E8L, and A30L |
| | proteins from MPXV |
| 3 | L1R, A27L, A33R, B5R, D8L, and A28L |
| | proteins from VACV |
| 4 | A2L, A3L, A3R, H3L, I1L, and/or A13 |
| | proteins from MPXV, and/or a |
| | VACV homolog thereof |

Methods of identifying protein homologs across species, e.g., the orthopoxviruses described herein, are known to one of ordinary skill in the art. For example, it will be understood by one of ordinary skill in the art that the MPXV M1R, A29L, A35R, B6R, E8L, and A30L proteins are the MPXV homologs of the VACV L1R, A27L, A33R, B5R, D8L, and A28L proteins, respectively. The amino acid sequences of the viral antigens described herein, e.g., in Table 1, are available from NCBI GenBank. For example, the sequences of the MPXV antigens are available under NCBI GenBank accession no. ON563414, and the sequences of the VACV antigens are available under NCBI GenBank accession no. DQ121394.1.

The terms “homolog” and “ortholog” are used interchangeably throughout the disclosure.

In embodiments, one or more of the antigens described herein has higher reactivity with serum from an infected recoveree, e.g., from MPXV infection, as compared to serum from a vaccinee, e.g., vaccinated with a VACV antigen, For example, studies by Cohn et al., medRxiv doi.org/10.1101/2023.03.07.23286701 (2023) and Yefet et al., iScience 26: 105957 (2023) demonstrated that recent MPXV infection (within 20-102 days) induced higher serum antibody responses to MPXV antigens A29L, A35R, A30L, and H3L, and to the VACV antigens A27L, B18R, and A33R, as compared to vaccinees. In embodiments, the immunoassay measures levels of antibody biomarkers that bind A29L, A35R, A30L, H3L, A27L, B18R, A33R, or combinations thereof. In embodiments, the invention provides a method identifying an individual as an infected recoveree or a vaccinee based on the measured antibody biomarker levels. In embodiments, for any given homolog pair, positive detection of the MPXV homolog and no detection of the VACV homolog is indicative of recent infection. In embodiments, for any given homolog pair, positive detection of the VACV homolog and no detection of the MPXV homolog is indicative of smallpox vaccination. Because smallpox has been effectively eradicated, detection of smallpox infection is unlikely. In embodiments, positive detection for both homologs is indicative of either infection or vaccination. In embodiments, a high ratio of signal or calculated concentration from a MPXV homolog to a VACV homolog indicates recent infection. In embodiments, a high signal or calculated concentration ratio from a VACV homolog to a MPXV homolog indicates vaccination. In embodiments, the signal or calculated concentration ratio of MPXV to VACV homolog in an infected subject is 1.5-fold, 2-fold, 3-fold, 4-fold, 5-fold, or greater than 5-fold. In embodiments, the signal ratio of VACV to MPXV homolog in a vaccinated subject is 1.5-fold, 2-fold, 3-fold, 4-fold, 5-fold, or greater than 5-fold.

In embodiments, the immunoassay detects an antibody biomarker that binds to a peptide antigen as the binding reagent. Peptide antigens are short peptides of a native, full-length protein that include the antibody binding epitope. Peptide antigens can be easier to produce and provide greater flexibility in performing an immunoassay to detect an antibody biomarker. Peptide antigens can also have higher specificity to the antibody biomarker compared with a full-length viral protein or domain described herein. In embodiments, an immunoassay utilizing a peptide antigen as the binding reagent has reduced cross-reactivity with antibody biomarkers for a different virus that are present in the sample. For example, an immunoassay utilizing an MPXV peptide antigen can have reduced cross-reactivity for antibodies that may be present in a subject due to vaccination with a VACV antigen. In embodiments, the peptide antigen is a fragment of a viral antigen, e.g., an MPXV or VACV antigen described herein. In embodiments, the peptide antigen comprises an immunodominant region (IDR) of a viral antigen. In embodiments, the peptide antigen comprises about 10 to about 100 amino acids. In embodiments, the peptide antigen comprises about 20 to about 80 amino acids. In embodiments, the peptide antigen comprises about 30 to about 60 amino acids. In embodiments, the peptide antigen comprises about 40 to about 50 amino acids. In embodiments, the peptide antigen is a fragment of any of the antigens shown in Table 1.

In embodiments, the method is a multiplexed immunoassay capable of simultaneously detecting and/or quantifying the amounts of one or more antibody biomarkers that bind to an orthopoxvirus antigen, e.g., MPXV antigen and/or VACV antigen. As discussed herein, a method that is capable of simultaneously assessing immune response against a panel of viral antigens from different viruses can advantageously allow an infection to be correctly and efficiently diagnosed in a single assay run and with a single patient sample. Such a method can also be useful for assessing a subject's immune response to different virus infections and/or determining the prior infection and/or vaccination status of a subject. In embodiments, the multiplexed immunoassay simultaneously detects and/or quantifies one or more antibody biomarkers that bind to a panel comprising any of the antigens shown in Table 1.

In embodiments, the multiplexed immunoassay comprises contacting the sample with a panel comprising at least two viral antigens. In embodiments, each viral antigen is immobilized or capable of being immobilized on a distinct binding domain on a surface. In embodiments, the panel of viral antigens comprise at least two of the antigens shown in Table 1, e.g., at least two of the antigens from Group 2 and/or 3 of Table 1.

In embodiments, the multiplexed immunoassay comprises: (a) forming a first binding complex comprising a first viral antigen and a first antibody biomarker; (b) measuring the concentration of the first antibody biomarker in the binding complex; and (c) repeating steps (a) and (b) for one or more additional antibody biomarkers, wherein each antibody biomarker binds to a different viral antigen of a panel of viral antigens. In embodiments, each of steps (a) and (b) is performed for each antibody biomarker in parallel. In embodiments, each viral antigen of the panel of viral antigens is immobilized to a surface. In embodiments, each viral antigen is capable of being immobilized to a surface. In embodiments, the panel of viral antigens are immobilized or capable of being immobilized on the same surface. In embodiments, the surface comprises a plurality of distinct binding domains, and each viral antigen is immobilized or capable of being immobilized on a distinct binding domain. In embodiments, the panel of viral antigens are immobilized on one or more surfaces. In embodiments, each viral antigen in the panel of viral antigens is immobilized on a separate surface. In embodiments, the panel of viral antigens comprise at least two of the antigens shown in Table 1, e.g., at least two of the antigens in Groups 2 and/or 3 of Table 1.

In embodiments, the immunoassay is a multiplexed immunoassay capable of simultaneously detecting and/or quantifying at least two antibody biomarkers in a sample, wherein each of the at least two antibody biomarkers is independently capable of binding to a viral antigen described herein, e.g., any of the antigens shown in Table 1, e.g., at least two of the antigens in Groups 2 and/or 3 of Table 1. In embodiments, the multiplexed immunoassay is capable of simultaneously detecting and/or quantifying two, three, four, five, or more than five antibody biomarkers in the biological sample, wherein each antibody biomarker is independently capable of binding to a viral antigen described herein. In embodiments, the multiplexed method comprising quantifying a combination of the antibody biomarkers provided herein has improved sensitivity and/or dynamic range, compared to a method in which only a single antibody biomarker is quantified. For example, a multiplexed method using a panel of viral antigens can provide earlier and more sensitive detection compared to a method that detects a single biomarker, since responses to each viral antigen may vary between individuals. Moreover, the ability to simultaneously measure antibody responses against multiple similar viruses, e.g., the MPXV isolate that emerged in the 2022 monkeypox outbreak (MPXV_USA_2022_MA001) and earlier MPXV isolates, improves understanding of how an individual's prior exposure to similar viruses affects the individual's response to a newly emerged virus of interest.

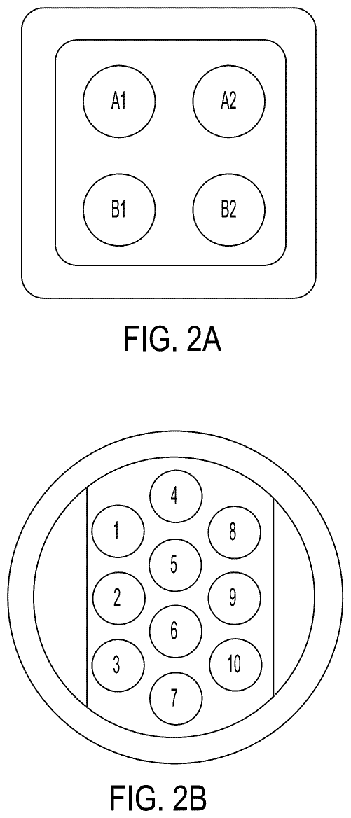

In embodiments, the panel of viral antigens, e.g., VACV and/or MPXV antigens, is immobilized on a surface. In embodiments, each viral antigen is immobilized on a distinct binding domain. In embodiments, the surface comprises a well of a multi-well assay plate. In embodiments, the surface, e.g., well of the multi-well assay plate, comprises four distinct binding domains (“Spots”), e.g., as shown in FIG. 2A. In embodiments, the surface, e.g., well of the multi-well assay plate, comprises ten distinct binding domains (“Spots”), e.g., as shown in FIG. 2B. In embodiments, the panel of viral antigens is immobilized on a surface comprising Spots 1-10 as shown in FIG. 2B, wherein each viral antigen is immobilized in the Spots as shown in Table 2. In embodiments, the panel of viral antigens is immobilized in the Spots as shown in Panel 1 of Table 2. In embodiments, the panel of viral antigens is immobilized in the Spots as shown in Panel 2 of Table 2. In embodiments, the panel of viral antigens is immobilized in the Spots as shown in Panel 3 of Table 2.

TABLE 2
Arrangement of Viral Antigen Panels in FIG. 2B Spots

| Panel 1 | | Panel 2 | | Panel 3 | |
|---|---|---|---|---|---|
| Spot # | Antigen | Spot # | Antigen | Spot # | Antigen |
| 1 | VACV A27L | 1 | VACV A28 | 1 | VACV A27L |
| 2 | VACV A33R | 2 | BSA | 2 | VACV A33R |
| 3 | VACV B5R | 3 | VACV L1R | 3 | VACV B5R |
| 4 | VACV D8L | 4 | BSA | 4 | VACV D8L |
| 5 | BSA | 5 | BSA | 5 | VACV L1R |
| 6 | BSA | 6 | BSA | 6 | MPXV M1R |
| 7 | MPXV E8L | 7 | BSA | 7 | MPXV E8L |
| 8 | MPXV B6R | 8 | MPXV M1R | 8 | MPXV B6R |
| 9 | MPXV A35R | 9 | BSA | 9 | MPXV A35R |
| 10 | MPXV A29L | 10 | MPXV A30L | 10 | MPXV A29L |

BSA: bovine serum albumin

In embodiments, the immunoassay method comprises: (a) contacting the sample with a surface comprising ten distinct binding domains; (b) forming a binding complex in the binding domain comprising a viral antigen and a biomarker that binds to the viral antigen; and (c) measuring the concentration of the biomarker in the binding complex. In embodiments, the viral antigens in the ten distinct binding domains are arranged as shown in Panel 1 of Table 2. In embodiments, the viral antigens in the ten distinct binding domains are arranged as shown in Panel 2 of Table 2. In embodiments, the viral antigens in the ten distinct binding domains are arranged as shown in Panel 3 of Table 2. In embodiments, the biomarker is IgG, IgA, IgM, or combination thereof. In embodiments, the biomarker is an IgG, IgA, and/or IgM from a human, NHP, mouse, or combination thereof. In embodiments, the concentration of the biomarker is measured by contacting the binding complex with a detection reagent that specifically binds IgG, IgA, or IgM as described herein. In embodiments, the immunoassay method is a classical serology assay. In embodiments, the immunoassay method is a bridging serology assay. In embodiments, the immunoassay method is a competitive serology assay. In embodiments, the immunoassay is a multiplexed immunoassay capable of simultaneously detecting and/or quantifying the one or more biomarkers that bind to the viral antigens.

The biomarkers described herein can be measured using a number of techniques available to a person of ordinary skill in the art, e.g., direct physical measurements (e.g., mass spectrometry) or binding assays (e.g., immunoassays, agglutination assays and immunochromatographic assays). Exemplary methods are described in, e.g., U.S. Publication No. 2022/0003766 and U.S. Publication No. 2021/0349104.

Exemplary binding assay methods include sandwich and competitive immunoassays, as described herein. Examples of sandwich immunoassays are described in U.S. Pat. Nos. 4,168,146 and 4,366,241. Examples of competitive immunoassays are described, e.g., in U.S. Pat. Nos. 4,235,601; 4,442,204; and 5,208,535.

Multiplexed assay formats are described, e.g., in US 2022/0003766; US 2021/0349104; US 2003/0113713; US 2003/0207290; US 2004/0022677; US 2004/0189311; US 2005/0052646; US 2005/0142033; US 2006/0069872; US 2021/0349104; US 2022/0003766; U.S. Pat. Nos. 5,807,522; 6,110,426; 6,977,722; 7,842,246; 10,189,023; and 10,201,812.

The methods herein can be conducted in a single assay chamber, such as a single well of an assay plate. The methods herein can also be conducted in an assay chamber of an assay cartridge as described herein. The assay modules (e.g., assay plates or assay cartridges), methods, and apparatuses for conducting assay measurements suitable for the present invention, are described, e.g., in U.S. Pat. Nos. 8,343,526; 9,731,297; 9,921,166; 10,184,884; 10,281,678; 10,272,436; US 2004/0022677; US 2004/0189311; US 2005/0052646; US 2005/0142033; US 2018/0074082; and US 2019/0391170.

Binding Reagent

In embodiments, the viral antigen of the immunoassay described herein, e.g., any of the antigens shown in Table 1, is a “binding reagent” of the immunoassay. In embodiments, the binding reagent is immobilized in a binding domain prior to being contacted with a sample comprising the biomarker(s) of interest. In embodiments, the binding reagent binds to an antibody biomarker to form a binding complex in the binding domain. In embodiments, the binding reagent is immobilized in a binding domain prior to, during, or after formation of the binding complex. In embodiments, the binding complex further comprises a detection reagent as described herein. In embodiments where the method is a multiplexed immunoassay method, at least two binding reagents are immobilized, each in a distinct binding domain. In embodiments, the at least two binding reagents are immobilized in their respective binding domains prior to, during, or after formation of the binding complexes as described herein. In embodiments, each binding domain comprises a targeting agent capable of binding to a targeting agent complement, wherein the targeting agent complement is connected to a linking agent, and each binding reagent comprises a supplemental linking agent capable of binding to the linking agent. Targeting agents, targeting agent complements, linking agents, and supplemental linking agents can simplify the process of immobilizing different binding reagents onto the surface.

In embodiments, the targeting agent and targeting agent complement, and the linking agent and supplemental linking agent, are each two members of a binding partner pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the targeting agent and targeting agent complement are cross-reactive moieties, e.g., thiol and maleimide or iodoacetamide; aldehyde and hydrazide; or azide and alkyne or cycloalkyne. In embodiments, the targeting agent is biotin, and the targeting agent complement is avidin or streptavidin. In embodiments, the linking agent is avidin or streptavidin, and the supplemental linking agent is biotin. In embodiments, the targeting agent and targeting agent complement are complementary oligonucleotides. In embodiments, the targeting agent complement is streptavidin, the targeting agent is biotin, and the linking agent and the supplemental linking agent are complementary oligonucleotides.

In embodiments, a bridging agent, which is a binding partner of both the linking agent and the supplemental linking agent, bridges the linking agent and supplemental linking agent, such that the binding reagents, each bound to its respective targeting agent complement, are contacted with the binding domains, and bind to their respective targeting agents via the bridging agent, the targeting agent complement on each of the binding reagents, and the targeting agent on each of the binding domains.

In embodiments, each binding domain is an element of an array of binding elements. In embodiments, the binding domains are on a surface. In embodiments, the surface is a plate. In embodiments, the surface is a well in a multi-well plate. In embodiments, the array of binding elements is located within a well of a multi-well plate. In embodiments, the array of binding elements are arranged as shown in FIG. 2A or 2B. Non-limiting examples of plates include the MSD® SECTOR™ and MSD QUICKPLEX® assay plates, e.g., MSD® GOLD™ 96-well Small Spot Streptavidin plate. In embodiments, the surface is a particle. In embodiments, the particle comprises a microsphere. In embodiments, the particle comprises a paramagnetic bead. In embodiments, each binding domain is positioned on one or more particles. In embodiments, the particles are in a particle array. In embodiments, the particles are coded to allow for identification of specific particles and distinguish between each binding domain. In embodiments, the surface is an assay cartridge surface. In embodiments, each binding domain is positioned in a distinct location on the assay cartridge surface.

Detection

In embodiments, the immunoassay method comprises detecting the binding complex described herein comprising the binding reagent (i.e., the viral antigen) and antibody biomarker. In embodiments, the binding complex further comprises a detection reagent. In embodiments, the detecting comprises detecting the detection reagent.

In embodiments, the method comprises forming the binding complex by contacting the sample comprising the antibody biomarker with: (i) the binding reagent and (ii) the detection reagent simultaneously or substantially simultaneously. In embodiments, the method comprises forming the binding complex by sequentially contacting the sample comprising the antibody biomarker with: first, the binding reagent, and second, the detection reagent. In embodiments, the method comprises forming the binding complex by sequentially contacting the sample comprising the antibody biomarker with: first, the detection reagent, and second, the binding reagent.

In embodiments, the surface comprises a binding reagent immobilized thereon, e.g., one or more binding reagents each immobilized in a distinct binding domain on the surface. In embodiments, the method comprises contacting the sample comprising the antibody biomarker with (i) the surface comprising the binding reagent and (ii) the detection reagent simultaneously or substantially simultaneously. In embodiments, the method comprises sequentially contacting the sample comprising the antibody biomarker with: first, the surface comprising the binding reagent and second, the detection reagent. In embodiments, the method comprises sequentially contacting the sample comprising the antibody biomarker with: first, the detection reagent and second, the surface comprising the binding reagent.

In embodiments, the detection reagent is an antibody, antigen, ligand, receptor, oligonucleotide, hapten, epitope, mimotope, or aptamer. In embodiments, the detection reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, detection reagent comprises at least one heavy or light chain CDR of an antibody. In embodiments, the detection reagent comprises at least two CDRs from one or more antibodies. In embodiments, the detection reagent is an antibody or antigen-binding fragment thereof.

In embodiments, the detection reagent comprises a detectable label. In embodiments, detecting the antibody biomarker comprises detecting the detectable label. In embodiments, the detectable label is detected by light scattering, optical absorbance, fluorescence, luminescence, chemiluminescence, electrochemiluminescence (ECL), bioluminescence, phosphorescence, radioactivity, magnetic field, or combination thereof. In embodiments, the detectable label comprises an electrochemiluminescence label. In embodiments, the detectable label comprises ruthenium. In embodiments, measuring the concentration of the biomarkers comprises measuring the presence and/or amount of the detectable label by electrochemiluminescence. In embodiments, the measuring of the detectable label comprises measuring an electrochemiluminescence signal.

In embodiments, detection reagent comprises a nucleic acid probe. In embodiments, the immunoassay further comprises binding the nucleic acid probe to a template oligonucleotide and extending the nucleic acid probe to form an extended sequence. In embodiments, the extended sequence binds to an anchoring reagent immobilized on the surface comprising the viral antigen. In embodiments, the antibody biomarker is detected and/or quantified by detecting or quantifying the amount of extended sequence bound to the surface. In embodiments, the surface is contacted with a labeled probe that binds to the extended sequence, wherein the labeled probe comprises a detectable label.

In embodiments, the binding complex comprises the binding reagent, the antibody biomarker, a first detection reagent, and a second detection reagent. In embodiments, the first detection reagent comprises a first nucleic acid probe, and the second detection reagent comprises a second nucleic acid probe. In embodiments, the immunoassay method further comprises binding the first and second nucleic acid probes to a template oligonucleotide and extending the second nucleic acid probe to form an extended sequence. In embodiments, the extended sequence binds to an anchoring reagent immobilized on the surface comprising the binding reagent. In embodiments, the antibody biomarker is detected and/or quantified by detecting or quantifying the amount of extended sequence bound to the surface. In embodiments, the surface is contacted with a labeled probe that binds to the extended sequence, wherein the labeled probe comprises a detectable label.

An exemplary immunoassay in which the detection reagent comprises a nucleic acid assay, as described in embodiments herein, comprises:
(a) contacting a biotinylated binding reagent and a biotinylated anchoring reagent with a surface comprising streptavidin or avidin, e.g., for about 30 minutes to about 2 hours at room temperature, or about 6 hours to about 12 hours at 4° C.; and optionally washing the surface to remove unbound binding reagent and/or anchoring reagent;
(b) contacting a sample comprising the analyte of interest (e.g., antibody biomarker described herein) with the surface, e.g., for about 1 hour to about 2 hours at about 20° C. to about 35° C. (e.g., about 27° C.); and optionally washing the surface to remove unbound analyte;
(c) contacting a detection reagent comprising a nucleic acid probe with the surface, e.g., for about 30 minutes to about 1 hour at about 20° C. to about 35° C. (e.g., about 27° C.), thereby forming a complex comprising the binding reagent, the analyte, and the detection reagent; and optionally washing the surface to remove unbound detection reagent;
(d) contacting a template oligonucleotide with the surface and ligating the template oligonucleotide to form a circular template, e.g., for about 10 minutes to about 30 minutes at about 20° C. to about 35° C. (e.g., about 27° C.), thereby hybridizing the nucleic acid probe to the circular template; and optionally washing the surface to remove excess template oligonucleotide;
(e) incubating the surface under conditions sufficient to perform rolling circle amplification, e.g., for about 5 minutes to about 30 minutes, thereby forming an extended sequence that binds to the anchoring reagent;
(f) contacting a labeled probe comprising a detectable label with the surface, e.g., for about 1 hour to about 2 hours at about 20° C. to about 35° C. (e.g., about 27° C.), thereby binding the labeled probe to the extended sequence; and optionally washing the surface to remove excess labeled probe; and
(g) measuring the amount of extended sequence by quantifying the amount of detectable label, thereby detecting and/or measuring the amount of analyte (e.g., antibody biomarker) in the sample.

In embodiments, the surface comprising the binding domains described herein comprises an electrode. In embodiments, the electrode is a carbon ink electrode. In embodiments, the measuring of the detectable label comprises applying a potential to the electrode and measuring electrochemiluminescence. In embodiments, applying a potential to the electrode generates an electrochemiluminescence signal. In embodiments, the strength of the electrochemiluminescence signal is based on the amount of detected analyte, e.g., antibody biomarker described herein, in the binding complex.

Detection methods are further described, e.g., in WO2014/165061; WO2014/160192; WO2015/175856; WO2020/180645; U.S. Pat. No. 9,618,510; U.S. Ser. No. 10/908,157; U.S. Ser. No. 10/114,015; US 2022/0003766; and US 2021/0349104.

Bridging Serology Assay

In embodiments, the immunoassay method is a bridging serology method. In a bridging serology assay, the binding complex comprises the binding reagent (i.e., viral antigen), the antibody biomarker, and a detection reagent described herein, wherein both the binding reagent and the detection reagent are an antigen that that is bound by the antibody biomarker. Since antibodies are typically bivalent, the antibody biomarker can bind both the binding reagent antigen and the detection reagent antigen. In embodiments, the detection reagent antigen is a viral antigen described herein. In embodiments, the detection reagent antigen and the binding reagent antigen each comprises a copy of the same viral protein. In embodiments, the detection reagent comprises any of the antigens shown in Table 1, e.g., in Groups 2 and/or 3 of Table 1.

In embodiments, the immunoassay method is a regular bridging serology assay. In a regular bridging serology assay, the antibody biomarker, binding reagent antigen, and detection reagent antigen are incubated together to form a complex where the antibody biomarker bivalently binds both the binding reagent antigen and the detection reagent antigen, e.g., a bridged complex. The incubation can be performed in any appropriate container, for example, in the well of a polypropylene plate, or in a chamber of an assay cartridge. In embodiments, the binding reagent antigen is conjugated to a biotin, and the bridged complex solution can be transferred to contact a surface comprising streptavidin, e.g., a streptavidin plate. In this embodiment, the biotin conjugated to the binding reagent antigen binds to the streptavidin plate, causing the entire bridged complex to be immobilized on the streptavidin plate.

In embodiments, antibody biomarkers are detected using a stepwise bridging serology assay. In a first step of a stepwise bridging serology assay, a binding reagent antigen is first immobilized on a surface. In embodiments, the binding reagent antigen is conjugated to biotin, and the plate is a streptavidin plate. In a second step, after the binding reagent antigen is immobilized on the surface, a solution containing the antibody biomarker is contacted with the surface, allowing the first bivalent position on the antibody biomarker to bind the binding reagent antigen. In a third step, the detection reagent antigen is then contacted with the surface, allowing the second bivalent position on the antibody to bind the detection reagent antigen. In this stepwise method, the bridging complex is formed stepwise on the surface, rather than forming the entire bridging complex before immobilization, as is done in the regular bridging assay. In the stepwise bridging assay, the surface may optionally be rinsed or washed between any of the steps.

In either of the regular bridging serology assay or stepwise bridging serology assay, a method may be used where the detectable label is not directly conjugated to the detection reagent antigen but is instead attached to the detection antigen reagent using a binding complex such as streptavidin/biotin or other binding pair. The advantage of using this method is that it is not necessary to prepare separately conjugated binding reagent antigen and detection reagent antigen. In a non-limiting example of this method, a biotin conjugated antigen is prepared. Some of this biotin conjugated antigen is then incubated with a detectable label conjugated with streptavidin. The binding of biotin to streptavidin causes the detectable label to become attached to the biotin conjugated antigen, creating a detection reagent antigen comprising a detectable label as follows:
Antigen-biotin-streptavidin-detectable label

In embodiments, additional free biotin is added to the antigen-detectable label reagent to fully occupy the streptavidin binding sites and prevent other biotin conjugates from binding to the antigen-detectable label reagent. An additional amount of the biotin conjugated antigen, which is not attached to a detectable label, is then used as the binding reagent antigen. Binding reagent antigen and detection reagent antigen prepared in this way may be used in any of the immunoassay methods described herein.

Classical Serology Assay

In embodiments, the antibody biomarker is detected using a classical serology assay. In a classical serology assay, the binding reagent antigen (i.e., viral antigen) is bound by the antibody biomarker to form a binding complex, and after the antibody biomarker is bound by the binding reagent antigen, the binding complex is contacted with a detection reagent antibody that binds the antibody biomarker.

In embodiments, the detection reagent antibody is an anti-human antibody that binds human antibody biomarkers. In embodiments, the detection reagent antibody is an anti-NHP antibody that binds NHP antibody biomarkers, an anti-mouse antibody that binds mouse antibody biomarkers, or an anti-rat antibody that binds rat antibody biomarkers. In embodiments, the detection reagent is an antibody or antigen-binding fragment that specifically binds IgG (e.g., IgG1, IgG2, IgG3, and IgG4), IgA, IgE, or IgM. Antibodies and fragments thereof that specifically bind to antibody biomarkers are known to one of ordinary skill in the art. In embodiments, the detection reagent antibody is an anti-human IgG, an anti-human IgM, or an anti-human IgA antibody. In embodiments, the detection reagent antibody is an anti-NHP IgG, IgM, or IgA antibody, an anti-mouse IgG, IgM, or IgA antibody, or an anti-rat IgG, IgM, or IgA antibody.

Competitive Serology Assay

In embodiments, the antibody biomarker is detected using a competitive serology assay (also termed a neutralization serology assay). In general terms, a competitive assay, e.g., a competitive immunoassay or a competitive inhibition assay, an analyte (e.g., an antibody biomarker described herein) and a competitor detection reagent compete for binding to a binding reagent (e.g., a viral antigen described herein). In such assays, the analyte is typically indirectly measured by directly measuring the competitor. As used herein, “competitor” refers to a compound capable of binding to the same binding reagent as an analyte, such that the binding reagent can only bind either the analyte or the competitor, but not both. In embodiments, competitive assays are used to detect and measure analytes that are not capable of binding more than one binding reagents, e.g., small molecule analytes or analytes that do not have more than one distinct binding sites. Examples of competitive immunoassays include those described in U.S. Pat. Nos. 4,235,601; 4,442,204; and 5,028,535.

In embodiments, the binding reagent is a viral antigen that is bound by the antibody biomarker and by a competitor. In embodiments, the competitor is a substance that binds a specific region of the viral antigen. In embodiments, the competitor is a recombinant antibody or antigen-binding fragment thereof that binds specifically to an epitope of the viral antigen, e.g., a neutralizing epitope. In embodiments, the competitor is a monoclonal antibody against an epitope of the viral antigen, e.g., a neutralizing epitope. In embodiments, the competitor comprises a detectable label described herein. In embodiments, a competitive serology assay as described herein is used to assess a potential protective serological response, e.g., the ability of the immune response to block binding of a viral antigen to its host cell receptor.

Calibration and Control Reagents

In embodiments, the immunoassay described herein further comprises measuring the concentration of one or more calibration reagents. In embodiments, a calibration reagent comprises a known concentration of a biomarker described herein. In embodiments, the calibration reagent comprises a mixture of known concentrations of multiple biomarkers. Measurement of calibration reagents is known in the art and further described, e.g., in US 2021/0349104 and US 2022/0003766.

In embodiments, the immunoassay described herein further comprises measuring the concentration of one or more control reagents. In embodiments, the control reagent is a positive control. In embodiments, the positive control comprises an antigen for which an antibody is known or expected to be present in the biological sample. In embodiments, the positive control comprises an antigen from a prevalent influenza strain, to which most subjects are expected to have antibodies. In embodiments, the positive control is an antigen from the H1 Michigan influenza virus. In embodiments, the positive control antigen is immobilized in a binding domain of the surface described herein. In embodiments, the immunoassay described herein further comprises measuring the total levels of a particular antibody, e.g., total IgG, IgA, or IgM, from the subject.

In embodiments, the control reagent is a negative control. In embodiments, the negative control comprises an antigen for which no antibodies are expected to be present in the biological sample. In embodiments, the negative control comprises a substance obtained from a non-human subject, and the biological sample is obtained from a human subject. In embodiments, the negative control comprises bovine serum albumin (BSA). In embodiments, the negative control, e.g., BSA, is immobilized in a binding domain of the surface described herein.

Exemplary Assay Methods

An exemplary multiplexed classical or bridging serology assay for detecting antibody biomarkers against MPXV and/or VACV antigens, and/or an exemplary multiplexed competitive serology assay detecting human neutralizing antibodies (also known as blocking antibodies) against MPXV and/or VACV antigens, as described in embodiments herein, comprises:

1. Preparation of assay plate. In embodiments, the assay plate is a 384-well assay plate. In embodiments, the assay plate is a 96-well assay plate. In embodiments, each well comprises four distinct binding domains, e.g., as shown in FIG. 2A. In embodiments, each well comprises ten distinct binding domains, e.g., as shown in FIG. 2B.

In embodiments, each well of the assay plate comprises ten distinct binding domains, wherein each binding domain comprises an immobilized viral antigen, e.g., MPXV and/or VACV antigen as described herein, e.g., as shown in Table 1. In embodiments, the viral antigens are immobilized on a surface comprising Spots 1-10 as shown in FIG. 2B, wherein the viral antigens are immobilized in the Spots as shown in Table 2, e.g., any of Panels 1, 2, or 3 of Table 2.

In embodiments, about 1 μL to about 200 μL, about 3 μL to about 150 μL, about 5 μL to about 100 μL, about 10 μL to about 90 μL, about 15 μL to about 80 μL, about 20 μL to about 70 L, about 30 μL to about 60 μL, about 50 μL, or about 150 μL of a blocking solution to each well of the plate. In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

2. Preparation of reagents. In embodiments, the assay comprises measuring the amount of one or more calibration reagents. Calibration reagents are further described herein. In embodiments, the assay comprises measuring the amount of multiple calibration reagents, e.g., at least 3, at least 4, at least 5, at least 6, at least 7, at least 8, at least 9, or at least 10 calibration reagents. In embodiments, the assay comprises generating a standard curve from the multiple calibration reagents. In embodiments, the assay comprises diluting a concentration reagent to provide multiple calibration reagents comprising a range of concentrations. In embodiments, the calibration reagent is diluted 1:10, 1:20, 1:30, 1:40, 1:50, 1:60, 1:70, 1:80, 1:90, 1:100, 1:140, 1:160, 1:200, 1:300, 1:400, 1:500, 1:600, 1:700, 1:800, 1:900, 1:1000, 1:1500, 1:2000, 1:2500, 1:3000, 1:3500, 1:4000, 1:4500, 1:5000, 1:5500, 1:6000, 1:6500, 1:7000, 1:7500, 1:8000, 1:8500, 1:9000, 1:9500, 1:10000, 1:20000, 1:30000, 1:40000, or 1:50000 to provide multiple concentrations of the calibration reagent. Calibration reagents are further described herein.

In embodiments, the assay comprises measuring the amount of one or more control reagents. In embodiments, the control reagent comprises a known quantity of IgG and/or IgM against the specific viral antigens in the assay, e.g., the MPXV and/or VACV antigens described herein, e.g., in Table 1. Control reagents are further described herein.

Examples of samples, e.g., biological samples, are provided herein. In embodiments, the sample is diluted about 2-fold, about 5-fold, about 10-fold, about 20-fold, about 30-fold, about 40-fold, about 50-fold, about 60-fold, about 70-fold, about 80-fold, about 90-fold, about 100-fold, about 250-fold, about 500-fold, about 750-fold, about 1000-fold, about 1500-fold, about 2000-fold, about 2500-fold, about 3000-fold, about 3500-fold, about 4000-fold, about 4500-fold, or about 5000-fold for use in the assay.

3. Addition of samples and reagents. In embodiments, the assay plate is washed at least once, at least twice, at least three times, at least four times, or at least five times with a wash buffer after incubation with the blocking solution. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, after the washing, the sample, one or more calibration reagents, and one or more control reagents are added to their respectively designated wells of the plate. In embodiments, about 5 μL to about 50 μL, about 10 μL to about 40 μL, about 20 μL to about 30 μL, about 15 μL, about 25 μL, or about 50 μL of the sample, calibration reagent, or control reagent is added to each well.

In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated while shaken at about 500 rpm to about 3000 rpm, about 800 rpm to about 2000 rpm, about 1000 rpm to about 1800 rpm, about 500 rpm to about 1000 rpm, or about 1200 rpm to about 1600 rpm. In embodiments, the plate is incubated for about 10 minutes to about 12 hours, or about 30 minutes to about 8 hours, or about 45 minutes to about 6 hours, or about 1 hour, or about 4 hours. In embodiments, the plate is incubated at about room temperature (e.g., about 22° C. to about 28° C.) while shaken at about 1500 rpm for about 4 hours. In embodiments, the plate is incubated at about room temperature (e.g., about 22° C. to about 28° C.) while shaken at about 700 rpm for about 1 hour.

4. Addition of detection reagent. In embodiments, the detection reagent (e.g., detection reagent antibody, detection reagent antigen, or competitor detection reagent for classical, bridging, and competitive serology assays, respectively) is diluted from a stock solution of detection reagent to obtain a solution comprising a working concentration of detection reagent. Detection reagents are further described herein.

In embodiments, the assay plate is washed at least once, at least twice, at least three times, at least four times, or at least five times with a wash buffer after incubation with the sample, calibration reagent, or control reagent. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, after the washing, the detection reagent solution for the classical or bridging serology assay, or the competitor solution for the competitive serology assay, is added to each well of the plate. In embodiments, about 5 μL to about 50 μL, about 10 μL to about 40 μL, about 10 μL to about 20 μL, about 20 μL to about 30 μL, about 15 μL, about 25 μL, or about 50 μL of the detection reagent solution or the competitor solution is added to each well.

In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated while shaken at about 500 rpm to about 3000 rpm, about 800 rpm to about 2000 rpm, about 1000 rpm to about 1800 rpm, about 500 rpm to about 1000 rpm, or about 1200 rpm to about 1600 rpm. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) while shaken at about 1500 rpm for about 1 hour. In embodiments, the plate is incubated at about room temperature (e.g., about 22° C. to about 28° C.) while shaken at about 700 rpm for about 1 hour.

5. Addition of read buffer. In embodiments, the assay plate is washed at least once, at least twice, at least three times, at least four times, or at least five times with a wash buffer after incubation with the detection reagent. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, the read buffer is added to each well of the plate. Read buffers are further described herein. In embodiments, about 5 μL to about 200 μL, about 5 μL to about 150 μL, about 5 μL to about 100 μL, about 10 μL to about 80 μL, about 20 μL to about 60 μL, about L, about 50 μL, about 100 μL, or about 150 μL of the read buffer is added to each well.

In embodiments, the assay comprises reading the plate, e.g., on a plate reader as described herein. In embodiments, the assay comprises reading the plate immediately following addition of the read buffer.

A further exemplary serology assay for detecting antibody biomarkers against MPXV and/or VACV antigens, as described in embodiments herein, comprises:
(a) mixing (i) a coating solution comprising a binding reagent bound to a linking agent and (ii) a detection reagent, wherein the binding reagent comprises an MPXV and/or VACV antigen, and wherein the detection reagent is a detection reagent antibody, detection reagent antigen, or competitor detection reagent as described herein and comprises a detectable label;
(b) contacting a surface with: (i) a sample comprising the antibody biomarker, (ii) a calibration reagent, or (iii) a control reagent, wherein the surface comprising one or more binding domains, wherein each binding domain comprises a targeting agent;
(c) contacting the surface with the mixture of (a); and
(d) measuring the amount of detectable label on the surface, thereby detecting and/or measuring the amount of the antibody biomarker. In embodiments, the MPXV and/or VACV antigens comprise any of the antigens shown in Table 1, e.g., Groups 2 and/or 3 of Table 1. In embodiments, the surface comprises Spots 1-10 as shown in FIG. 2B, wherein the viral antigens are immobilized in the Spots as shown in Table 2, e.g., any of Panels 1, 2, or 3 of Table 2.

A further exemplary serology assay for detecting antibody biomarkers against MPXV and/or VACV antigens, as described in embodiments herein, comprises:
(a) mixing (i) a biotinylated binding reagent and (ii) a detection reagent, wherein the binding reagent comprises an MPXV and/or VACV antigen, and wherein the detection reagent is a detection reagent antibody, detection reagent antigen, or competitor detection reagent as described herein and comprises a detectable label;
(b) contacting a surface with: (i) a sample comprising the antibody biomarker, (ii) a calibration reagent, or (iii) a control reagent, wherein the surface comprising one or more binding domains, wherein each binding domain comprises avidin or streptavidin;
(c) contacting the surface with the mixture of (a); and
(d) measuring the amount of detectable label on the surface, thereby detecting and/or measuring the amount of the antibody biomarker. In embodiments, the MPXV and/or VACV antigens comprise any of the antigens shown in Table 1, e.g., Groups 2 and/or 3 of Table 1. In embodiments, the surface comprises Spots 1-10 as shown in FIG. 2B, wherein the viral antigens are immobilized in the Spots as shown in Table 2, e.g., any of Panels 1, 2, or 3 of Table 2.

In embodiments, the surface is a multi-well plate. In embodiments, the assay further comprises a wash step prior to one or more of the assay steps. In embodiments, the wash step comprises washing the assay plate at least once, at least twice, at least three times, at least four times, or at least five times with a wash buffer. In embodiments, the assay plate is washed with at least about 10 μL, at least about 15 μL, at least about 20 μL, at least about 25 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer. In embodiments, the classical or bridging serology assay does not comprise a wash step prior to any of steps (a), (b), or (c). In embodiments, the competitive serology assay does not comprise a wash step prior to any of steps (a), (b), or (c).

In embodiments, prior to step (a), a blocking solution is added to the plate to reduce non-specific binding of the coating solution or the biotinylated binding reagent to the surface. In embodiments, about 50 μL to about 250 μL, about 100 μL to about 200 μL, or about 150 μL of blocking solution is added per well of the plate. In embodiments, the plate is incubated at about 15° C. to about 30° C., about 18° C. to about 28° C., about 20° C. to about 26° C., or about 22° C. to about 24° C. In embodiments, the plate is incubated while shaken at about at about 500 rpm to about 2000 rpm, about 600 rpm to about 1500 rpm, or about 700 rpm to about 1000 rpm. In embodiments, the method comprises incubating the blocking solution on the plate for about 10 minutes to about 4 hours, about 20 minutes to about 3 hours, or about 30 minutes to about 2 minutes. In embodiments, the plate is incubated at about room temperature (e.g., about 22° C. to about 28° C.) while shaken at about 700 rpm for about 30 minutes to about 2 hours.

In embodiments comprising a coating solution, the assay further comprises, prior to step (a), mixing a linking agent connected to a targeting agent complement with a binding reagent comprising a supplemental linking agent, thereby forming the coating solution comprising the binding reagent bound to the linking agent. In embodiments, the method comprises forming about 200 μL to about 1000 μL, or about 300 μL to about 800 μL, or about 400 μL to about 600 μL of the coating solution. In embodiments, step (a) comprises incubating the linking agent and the binding reagent at about 15° C. to about 30° C., about 18° C. to about 28° C., about 20° C. to about 26° C., or about 22° C. to about 24° C. In embodiments, the method comprises forming about 500 μL of the coating solution by incubating about 300 μL of a solution comprising the linking agent and about 200 μL of a solution comprising the binding reagent, at about room temperature (e.g., about 22° C. to about 28° C.) for about 30 minutes. In embodiments, the incubating is performed without shaking. In embodiments, the assay further comprises contacting the coating solution with a stop solution (e.g., about 100 μL to about 500 μL, or about 150 μL to about 300 μL, or about 200 μL of a stop solution) to stop the binding reaction between the linking agent and supplemental linking agent. In embodiments, the coating solution and the stop solution are incubated for about 10 minutes to about 1 hour, about 20 minutes to about 40 minutes, or about 30 minutes. In embodiments, the coating solution and the stop solution are incubated at about 15° C. to about 30° C., about 18° C. to about 28° C., about 20° C. to about 26° C., or about 22° C. to about 24° C. In embodiments, the method further comprises, following incubation of the coating solution with the stop solution, diluting the coating solution using the stop solution, e.g., by 2-fold, 5-fold, 10-fold, or 20-fold, to a working concentration as described herein. In embodiments, the targeting agent and targeting agent complement comprise complementary oligonucleotides. In embodiments, the linking agent comprises avidin or streptavidin, and the supplemental linking agent comprises biotin.

In embodiments, about 10 μL to about 200 μL, about 5 μL to about 100 μL, about 10 μL to about 90 μL, about 15 μL to about 80 μL, about 20 μL to about 70 μL, about 30 μL to about 60 μL, or about 50 μL of the coating solution or a solution containing the biotinylated binding reagent are added to each well of the plate. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments, about 5 μL to about 50 μL, about 10 μL to about 40 μL, about 20 μL to about 30 μL, about 15 μL, about 25 μL, about 35 μL, or about 50 μL of the sample, calibration reagent, or control reagent are added to each well of the plate. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments where the assay is a classical or bridging serology assay, about 5 μL to about 50 μL, about 10 μL to about 40 μL, about 10 μL to about 20 μL, about 20 μL to about 30 μL, about 15 μL, about 25 μL, about 35 μL, or about 50 μL of the mixture comprising the binding reagent and the detection reagent to each well of the plate. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments where the assay is a competitive serology assay, about 5 μL to about 50 μL, about 10 μL to about 40 μL, about 10 μL to about 20 μL, about 20 μL to about 30 μL, about L, about 25 μL, about 35 μL, or about 50 μL of a solution comprising the ACE2 detection reagent are added to each well of the plate. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments, step (d) comprises adding a read buffer to each well of the plate. Read buffers are further described herein. In embodiments, about 5 μL to about 200 μL, about 5 μL to about 150 μL, about 5 μL to about 100 μL, about 10 μL to about 80 μL, about 20 μL to about 60 μL, about 40 μL, about 50 μL, about 100 μL, or about 150 μL of the read buffer is added to each well. In embodiments, the measuring comprises reading the plate, e.g., on a plate reader as described herein. In embodiments, the assay comprises reading the plate immediately following addition of the read buffer.

### Samples and Assay Devices

In embodiments, the biomarkers described herein are present in a sample. In embodiments, the sample is a biological sample. In embodiments, the biological sample comprises a mammalian fluid, secretion, or excretion. In embodiments, the sample is a purified mammalian fluid, secretion, or excretion. In embodiments, the mammalian fluid, secretion, or excretion is whole blood, plasma, serum, sputum, lachrymal fluid, lymphatic fluid, synovial fluid, pleural effusion, urine, sweat, cerebrospinal fluid, ascites, milk, stool, a respiratory sample, bronchial/bronchoalveolar lavage, saliva, mucus, oropharyngeal swab, sputum, endotracheal aspirate, pharyngeal/nasal swab, throat swab, amniotic fluid, nasal secretions, nasopharyngeal wash or aspirate, nasal mid-turbinate swab, vaginal secretions, a surface biopsy, sperm, semen/seminal fluid, wound secretions and excretions, ear secretions or discharge, or an extraction, purification therefrom, or dilution thereof. In embodiments, the biological sample is diluted such that the assay signal is within the upper and lower detection limits of the assay. In embodiments, the biological sample is diluted to achieve a desired assay sensitivity. Further exemplary biological samples include but are not limited to physiological samples, samples containing suspensions of cells such as mucosal swabs, tissue aspirates, endotracheal aspirates, tissue homogenates, cell cultures, and cell culture supernatants.

In embodiments, the biological sample is a respiratory sample obtained from the respiratory tract of a subject. Examples of respiratory samples include, but are not limited to, bronchial/bronchoalveolar lavage, saliva, mucus, endotracheal aspirate, sputum, nasopharyngeal/nasal swab, throat swab, oropharyngeal swab and the like.

In embodiments, the biological sample is whole blood, serum, plasma, cerebrospinal fluid (CSF), urine, saliva, sputum, endotracheal aspirate, nasopharyngeal/nasal swab, bronchoalveolar lavage, or an extraction or purification therefrom, or dilution thereof. In embodiments, the biological sample is blood that has been dried and reconstituted. In embodiments, the biological sample is serum or plasma. In embodiments, the plasma is in EDTA, heparin, or citrate.

In embodiments, the biological sample is saliva. In embodiments, the biological sample is endotracheal aspirate. In embodiments, the biological sample is a nasal swab. In embodiments, the biomarkers described herein are present in higher amounts in certain bodily fluids (e.g., saliva) compared to others (e.g., throat swab). In embodiments, certain antibody biomarker levels, e.g., IgG (including subclasses thereof) and IgA, are substantially similar in blood and saliva of a subject. In embodiments, the ratio of antibody levels to different components from a virus are highly correlated in blood and saliva of a subject. In embodiments, the ratio of antibody levels to different components from a virus, e.g., the ratio of the antibody levels against the two different MPXV antigens is used to assess the immune response and/or clinical outcome of a subject infected with MPXV.

In embodiments, the biological sample is from an animal. In embodiments, the biological sample from an animal is useful for animal model studies, e.g., for vaccine and/or drug research and development, and/or to better understand disease progression and infection lethality. Exemplary animals that are useful for animal model studies include, but are not limited to, mouse, rat, rabbit, pig, NHP, and the like.

In embodiments, the biological sample is from a human or an animal subject. In embodiments, the subject is susceptible or suspected to be susceptible to infection by the viruses described herein. In embodiments, the subject is known or suspected to transmit the viruses described herein. Virus transmission may occur among the same species (e.g., human-to-human) or inter-species (e.g., bat-to-human). Non-limiting examples of animal subjects include domestic animals, such as dog, cat, horse, goat, sheep, donkey, pig, cow, chicken, duck, rabbit, gerbil, hamster, guinea pig, and the like; NHPs such as macaque, baboon, marmoset, gorilla, orangutan, chimpanzee, monkey, and the like; big cats such as tiger, lion, puma, leopard, snow leopard, and the like; and other mammals such as bats and pangolins. In embodiments, the biological sample is from a human, an NHP, a mouse, or a rat. In embodiments, the subject is a host that has been exposed to and/or infected by a virus as described herein. In embodiments, the biological ample comprises a plasma (e.g., in EDTA, heparin, or citrate) sample from a subject. In embodiments, the biological sample comprises a serum sample from a subject. In embodiments, the biological sample is from a healthy subject. In embodiments, the biological sample is from a subject known to never have been exposed to a virus described herein. In embodiments, the biological sample is from a subject known to be immune to a virus described herein. In embodiments, the biological sample is from a subject known to be infected with a virus described herein. In embodiments, the biological sample is from a subject suspected of having been exposed to a virus described herein. In embodiments, the biological sample is from a subject at risk of being exposed to a virus described herein. In embodiments, the virus is an orthopoxvirus, e.g., MPXV.

In embodiments, the sample is an environmental sample. In embodiments, the environmental sample is aqueous, including but not limited to, fresh water, drinking water, marine water, reclaimed water, treated water, desalinated water, sewage, wastewater, surface water, ground water, runoff, aquifers, lakes, rivers, streams, oceans, and other natural or non-natural bodies of water. In embodiments, the aqueous sample contains bodily solids or fluids (e.g., feces or urine) from subjects who have been exposed to or infected with a virus herein (e.g., an orthopoxvirus, e.g., MPXV). In embodiments, the environmental sample is from an air filtration device, e.g., air filters in a healthcare or long-term care facility or other communal places of gathering. Detection of a virus described herein (e.g., an orthopoxvirus, e.g., MPXV) in an environmental sample can provide early identification and/or tracing of an outbreak or potential outbreak, thereby allowing a more prompt and robust response. Moreover, detection of a biomarker, e.g., one or more antibody biomarkers that specifically binds a viral antigen (e.g., from an orthopoxvirus, e.g., MPXV) in an environmental sample can provide an estimation of the percentage of a population with detectable antibodies against the virus (i.e., seroconversion), which is useful for epidemiology studies. In embodiments, the sample comprises wastewater.

In embodiments where the sample comprises a liquid (e.g., endotracheal aspirate, saliva, blood, serum, plasma and the like), the sample is about 0.05 mL to about 50 mL, about 0.1 mL to about 10 mL, about 0.2 mL to about 5 mL, or about 0.3 mL to about 3 mL. In embodiments where the sample is solid or semi-solid (e.g., a swab such as a nasopharyngeal swab or oropharyngeal swab, mucus, sputum and the like), the sample is provided into a storage liquid of about 0.05 mL to about 50 mL, about 0.1 mL to about 10 mL, about 0.2 mL to about 5 mL, or about 0.3 mL to about 3 mL. In embodiments, the storage liquid is Viral Transport Medium (VTM), Amies transport medium, or sterile saline. In embodiments, the storage liquid comprises a reagent for inactivating live virus as described herein.

In embodiments, the sample is pretreated prior to being subjected to the methods provided herein. In embodiments, the sample is pretreated prior to being handled by, processed by, or in contact with laboratory and/or clinical personnel. In embodiments, pretreating the sample comprises subjecting the sample to conditions sufficient to inactivate live virus in the sample. Inactivation of live virus that may be present in the sample reduces the risk of infection of the laboratory and/or clinical personnel handling and/or processing the sample, e.g., by performing the methods described herein on the sample. In embodiments, pretreating the sample comprises heating the sample to at least 55° C., at least 56° C., at least 57° C., at least 58° C., at least 59° C., at least 60° C., at least 65° C., at least 70° C., at least 75° C., at least 80° C., at least 85° C., at least 90° C., at least 95° C., or at least 100° C. In embodiments, the sample is heated for about 10 minutes to about 4 hours, about 20 minutes to about 2 hours, or about 30 minutes to about 1 hour. In embodiments, the sample is heated to about 65° C. for at least 10 minutes. In embodiments, the sample is heated to about 65° C. for at least 30 minutes. In embodiments, the sample is heated to about 58° C. for at least 1 hour.

In embodiments, pretreating the sample comprises contacting the sample with an inactivation reagent. In embodiments, the inactivation reagent comprises a detergent, a chaotropic agent, a fixative, or a combination thereof. Non-limiting examples of detergents include sodium dodecyl sulfate and TRITON™ X-100. Non-limiting examples of chaotropic agents include guanidium thiocyanate, guanidium isothiocyanate, and guanidium hydrochloride. Non-limiting examples of fixatives include formaldehyde, formalin, paraformaldehyde, and glutaraldehyde. In embodiments, pretreating the sample comprises subjecting the sample to UV or gamma irradiation. In embodiments, pretreating the sample comprises subjecting the sample to a highly alkaline (e.g., above pH 10, above pH 11, or above pH 12) condition. In embodiments, pretreating the sample comprises subjecting the sample to a highly acidic (e.g., below pH 4, below pH 3, below pH 2) condition.

In embodiments, the sample is pretreated immediately after being collected, e.g., from a subject described herein. Sample collection methods are provided herein. In embodiments, the sample is pretreated while being transported to a facility, e.g., a laboratory, for processing and analyzing the sample, e.g., using the methods described herein. In embodiments, the sample is pretreated after arrival at a facility, e.g., a laboratory, for processing and analyzing the sample, e.g., using the methods described herein. In embodiments, the sample is pretreated prior to being stored. In embodiments, the sample is stored prior to processing and analysis, e.g., using the methods described herein. In embodiments, the sample is stored at about −80° C. to about 30° C., about −70° C. to about 25° C., about −60° C. to about 20° C., about −20° C. to about 15° C., about 0° C. to about 10° C., about 2° C. to about 8° C., or about 4° C. to about 12° C. Methods and conditions for storing the samples described herein are known to one of ordinary skill in the art.

As used herein, the term “exposure,” in the context of a subject being exposed to a virus, refers to the introduction of a virus into the subject's body. “Exposure” does not imply any particular amount of virus; introduction of a single viral particle into the subject's body can be referred to herein as an “exposure” to the virus. As used herein, the term “infection,” in the context of a subject being infected with a virus, means that the virus has penetrated a host cell and has begun to replicate, assemble, and release new viruses from the host cell. The term “infection” can also be used to refer to an illness or condition caused by a virus, e.g., monkeypox.

In embodiments, the biomarker is detectable in a subject immediately (e.g., within seconds) after the subject is exposed to the virus and/or infected with the virus, e.g., an orthopoxvirus such as MPXV. In embodiments, the biomarker is detectable in a subject within about 5 minutes to about 1 year, about 1 hour to about 9 months, about 6 hours to about 6 months, about 12 hours to about 90 days, about 1 day to about 60 days, about 2 days to about 50 days, about 3 days to about 40 days, about 4 days to about 30 days, about 5 days to about 28 days, about 6 days to about 25 days, about 7 days to about 22 days, or about 8 days to about 20 days after the subject is exposed to the virus and/or infected with the virus. In embodiments, the biomarker is detectable in a subject within about 5 minutes, about 1 hour, about 3 hours, about 6 hours, about 12 hours, about 1 day, about 2 days, about 3 days, about 4 days, about 5 days, about 6 days, about 7 days, about 10 days, about 14 days, about 21 days, about 1 month, about 2 months, about 3 months, about 6 months, about 1 year, or more than 1 year after the subject is exposed to the virus and/or infected with the virus. Different antibody biomarkers in the same subject may have a varying magnitude of change in response to virus exposure and/or infection. For some viral infections, the antibody biomarker IgG typically plateaus after 10 days of disease onset and persist (e.g., potentially signifying longer-term immunity); the antibody biomarkers IgA and IgM are detectable within 6 days of disease onset, peak around 10 days, and diminish after approximately 14 days (e.g., as part of the initial infection response). Different viruses can trigger biomarker responses at different times. The timing of producing the same biomarker type, e.g., IgM or IgG antibody, can also vary widely among different subjects. Thus, in embodiments, the multiplexed assays for a combination of biomarkers provided herein are used to determine or assess the response timing of each of the biomarkers.

In embodiments, the biological sample is obtained from a subject who has not been exposed to the virus, e.g., an orthopoxvirus such as MPXV. In embodiments, the biological sample is obtained from a subject immediately (e.g., within seconds) after the subject is known or suspected to be exposed to the virus. In embodiments, the biological sample is obtained from a subject within about 5 minutes to about 1 year, about 1 hour to about 9 months, about 6 hours to about 6 months, about 12 hours to about 90 days, 1 day to about 60 days, about 2 days to about 50 days, about 3 days to about 40 days, about 4 days to about 30 days, about 5 days to about 28 days, about 6 days to about 25 days, about 7 days to about 22 days, or about 8 days to about 20 days after the subject is known or suspected to be exposed to the virus. In embodiments, the biological sample is obtained from a subject within about 5 minutes, about 1 hour, about 3 hours, about 6 hours, about 12 hours, about 1 day, about 2 days, about 3 days, about 4 days, about 5 days, about 6 days, about 7 days, about 10 days, about 14 days, about 21 days, about 1 month, about 2 months, about 3 months, about 6 months, about 1 year, or more than 1 year after the subject is known or suspected to be exposed to the virus.

In embodiments, the biological sample is obtained from a subject prior to the subject showing any symptoms of a viral infection, e.g., by an orthopoxvirus such as MPXV. In embodiments, the biological sample is obtained from a subject immediately (e.g., within seconds) after the subject begins to show symptoms of a viral infection. In embodiments, the biological sample is obtained from a subject within about 5 minutes to about 1 year, about 1 hour to about 9 months, about 6 hours to about 6 months, about 12 hours to about 90 days, about 1 day to about 60 days, about 2 days to about 50 days, about 3 days to about 40 days, about 4 days to about 30 days, about 5 days to about 28 days, about 6 days to about 25 days, about 7 days to about 22 days, or about 8 days to about 20 days after the subject begins to show symptoms of a viral infection. In embodiments, the biological sample is obtained from a subject within about 5 minutes, about 1 hour, about 3 hours, about 6 hours, about 12 hours, about 1 day, about 2 days, about 3 days, about 4 days, about 5 days, about 6 days, about 7 days, about 10 days, about 14 days, about 21 days, about 1 month, about 2 months, about 3 months, about 6 months, about 1 year, or more than 1 year after the subject begins to show symptoms of a viral infection. Symptoms of infection by viruses described herein, e.g., an orthopoxvirus such as MPXV include, e.g., skin lesions.

In embodiments, the biological sample is obtained from a subject after the subject is diagnosed with a viral infection, e.g., by an orthopoxvirus such as MPXV. In embodiments, the biological sample is obtained from a subject after about 1 day, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 9 months, about 1 year, about 2 years, about 3 years, about 4 years, about 5 years, about 6 years, about 7 years, about 8 years, about 9 years, about 10 years, or more than 10 years after the subject is diagnosed with the viral infection.

In embodiments, the biological sample is obtained from a subject prior to the subject being administered with a vaccine or a treatment for the virus described herein, e.g., an orthopoxvirus such as MPXV. In embodiments, the biological sample is obtained from a subject immediately (e.g., within seconds) after a vaccine or a treatment is administered to the subject. In embodiments, the biological sample is obtained from a subject within about 12 hours to about 90 days, about 1 day to about 60 days, about 2 days to about 50 days, about 3 days to about 40 days, about 4 days to about 30 days, about 5 days to about 28 days, about 6 days to about 25 days, about 7 days to about 22 days, or about 8 days to about 20 days after a vaccine or a treatment is administered to the subject. In embodiments, the biological sample is obtained from a subject within about 5 minutes, about 1 hour, about 3 hours, about 6 hours, about 12 hours, about 1 day, about 2 days, about 3 days, about 4 days, about 5 days, about 6 days, about 7 days, about 10 days, about 14 days, about 21 days, about 1 month, about 2 months, about 3 months, about 6 months, about 1 year, or more than 1 year after a vaccine or a treatment is administered to the subject.

Sample Pooling

Samples may be obtained from a single source described herein, or may contain a mixture from two or more sources, e.g., pooled from one or more individuals who may have been exposed to or infected by a particular virus in a similar manner. For example, the individuals may live or have lived in the same household, visited the same location(s), and/or associated with the same people. In embodiments, samples are pooled from two or more, three or more, four or more, five or more, six or more, seven or more, eight or more, nine or more, ten or more, 15 or more, 20 or more, 25 or more, 30 or more, 40 or more, 50 or more, 100 or more, 150 or more, 200 or more, 300 or more, 400 or more, 500 or more, 1000 or more, 5000 or more, or 10000 or more individuals. For example, a “negative” result for an active viral infection from a pooled sample indicates that none of the individuals from the pooled sample have an active infection, which can significantly reduce the number of tests needed to test every individual in a population. In embodiments, the sample comprises a respiratory sample, e.g., bronchial/bronchoalveolar lavage, saliva, mucus, oropharyngeal swab, sputum, endotracheal aspirate, pharyngeal/nasal swab, throat swab, nasal secretion, or combination thereof. In embodiments, the sample comprises saliva. In embodiments, the sample comprises blood. In embodiments, the sample comprises serum or plasma. In embodiments, a “positive” result for an active viral infection in the pooled sample prompts or indicates a need for further testing using the methods and/or kits provided by the invention of individual samples comprised in the pool of samples. Sample pooling strategies are further described, e.g., in U.S. Publication No. 2022/0003766; U.S. Publication No. 2021/0349104; and U.S. Publication No. 2022/0003766.

Collection and Assay Devices

In embodiments, the biological sample is a liquid sample. In embodiments, the biological sample is in contact with a sample collection device. In embodiments, the sample collection device is an applicator stick. In embodiments, the sample collection device comprises an elongated handle (e.g., a rod or a rectangular prism) and a sample collection head configured to collect sample from a biological tissue (e.g., from a subject's nasal or oral cavity) or a surface. In embodiments, the sample collection head comprises an absorbent material (e.g., cotton) or a scraping blade. In embodiments, the sample collection device is a swab. In embodiments, the sample collection device is a tissue scraper. In embodiments, the sample collection device is capable of collecting a sample described herein that may contain analytes at a concentration too low to support an accurate or reliable analysis result.

In embodiments, the sample collection device or the liquid sample is contacted with an assay cartridge. Assay cartridges are further described in, e.g., U.S. Publication No. 2022/0003766 and U.S. Publication No. 2021/0349104. Assay cartridges may be used with assay cartridge readers known in the art. An exemplary assay cartridge reader is the MSD® Cartridge Reader instrument. Further exemplary assay cartridges and assay cartridge readers are described, e.g., in U.S. Pat. Nos. 9,921,166; 10,184,884; 9,731,297; 8,343,526; 10,281,678; 10,272,436; US 2018/0074082; and US 2019/0391170.

In embodiments, the method is performed in an assay plate. Assay plates are known in the art and described, e.g., in U.S. Publication No. 2022/0003766 and U.S. Publication No. 2021/0349104. Further exemplary assay plates are disclosed in, e.g., U.S. Pat. Nos. 7,842,246; 8,790,578; and 8,808,627. In embodiments, the assay plate result is read in a plate reader, e.g., the MESO® QUICKPLEX® or MESO® SECTOR® instruments.

In embodiments, the method is performed on a particle. Particles known in the art, e.g., as described in U.S. Publication No. 2022/0003766 and U.S. Publication No. 2021/0349104, can be used in conjunction with the methods and kits described herein. In embodiments, the particle comprises a microsphere.

Further exemplary devices for performing the methods herein include, but are not limited to, cassettes, measurement cells, dipsticks, reaction vessels, and assay modules described in, e.g., U.S. Pat. Nos. 8,298,934 and 9,878,323.

### Manual and Automated Embodiments

The methods herein can be performed manually, using automated technology, or both. Automated technology may be partially automated, e.g., one or more modular instruments, or a fully integrated, automated instrument. Exemplary automated systems and apparatuses are described in WO 2018/017156, WO 2017/015636, and WO 2016/164477. In embodiments, the methods herein are performed in an automated cartridge reader as described herein. Manual and automated systems for use with the methods and kits described herein are known in the art and described, e.g., in US 2021/0349104 and US 2022/0003766.

### Kits

In embodiments, the invention provides a kit for detecting one or more antibody biomarkers of interest in a sample, the kit comprising, in one or more vials, containers, or compartments: (a) a surface comprising a panel of antigens immobilized on one or more binding domains, wherein each binding domain comprises an antigen of the panel of antigens immobilized thereon, wherein the panel of antigens comprises a monkeypox virus (MPXV) protein, a vaccinia virus (VACV) protein, or combination thereof; and (b) one or more detection reagents, wherein each detection reagent comprises a detection antibody, a detection antigen, or a detection competitor. In embodiments, the antigens comprise any of the antigens shown in Table 1. In embodiments, the panel of antigens comprise any of the panels shown in Table 2.

In embodiments, the invention provides a kit for detecting one or more antibody biomarkers of interest in a sample, comprising, in one or more vials, containers, or compartments: (a) a viral antigen that specifically binds an antibody biomarker; and (b) a detection reagent that specifically binds the antibody biomarker. In embodiments, the kit further comprises a surface. In embodiments, the viral antigen is immobilized to the surface or capable of being immobilized to the surface. In embodiments, the viral antigens comprise an MPXV protein, a VACV protein, or combination thereof, e.g., any of the antigens shown in Table 1. In embodiments, the kit comprises a panel of viral antigens. In embodiments, each viral antigen of the panel of viral antigens is immobilized to the surface or capable of being immobilized to the surface. In embodiments, the panel of viral antigens comprises any of the panels shown in Table 2.

Antibody biomarkers and their binding partners, e.g., viral antigens, are described herein. In embodiments, the viral antigen is an MPXV and/or VACV antigen as described herein. In embodiments, the antibody biomarkers comprises IgG, IgA, IgM, or a combination thereof. In embodiments, the IgG, IgA, and/or IgM is from a human, NHP, mouse, rat, or combination thereof. In embodiments, the detection reagent specifically binds IgA, IgG, or IgM. In embodiments, the detection reagent is an antibody or antigen-binding fragment thereof. In embodiments, the detection reagent is a second copy of the viral antigen. In embodiments, the detection reagent is a competitor detection reagent as described herein.

In embodiments, the surface comprises a single assay plate. In embodiments, the surface comprises a multi-well assay plate, wherein each well comprises four distinct binding domains, e.g., as shown in FIG. 2A. In embodiments, the surface comprises a multi-well assay plate, wherein each well comprises ten distinct binding domains, e.g., as shown in FIG. 2B. In embodiments, the assay plate is a 96-well assay plate. In embodiments, the assay plate is a 384-well assay plate. In embodiments, the surface comprises one or more binding domains, wherein each binding domain comprises a viral antigen immobilized thereon. In embodiments, the surface comprises a well of an assay plate. In embodiments, each well of the assay plate comprises ten distinct binding domains, wherein each binding domain comprises an immobilized viral antigen, e.g., MPXV and/or VACV antigen as described herein. In embodiments, the viral antigens are immobilized on a surface comprising Spots 1-10 as shown in FIG. 2B, wherein the viral antigens are immobilized in the Spots as shown in Table 2, e.g., any of Panels 1, 2, or 3 of Table 2.

In embodiments, the surface comprises avidin or streptavidin. In embodiments, each binding reagent comprises biotin. In embodiments, the surface comprises a targeting agent. In embodiments, the kit further comprises a linking agent connected to a targeting agent complement. In embodiments, each binding reagent comprises a supplemental linking agent. In embodiments, the targeting agent and targeting agent complement comprise complementary oligonucleotides. In embodiments, the linking agent comprises avidin or streptavidin, and the supplemental linking agent comprises biotin. Targeting agents, targeting agent complements, linking agents, and supplemental linking agents are further described herein.

In embodiments, the invention provides a combination of any of the kits described herein. In embodiments, the combination of kits is provided as a single kit, comprising the components of each of the individual kits.

In embodiments, the binding reagent is a viral antigen, e.g., MPXV and/or VACV antigen as described herein. In embodiments, the detection reagent is an antibody or antigen-binding fragment thereof. In embodiments, the detection reagent described herein comprises a detectable label as described herein. In embodiments, the detection reagent comprises a nucleic acid probe as described herein. In embodiments, the kit comprises first and second detection reagents, and the first and second detection reagents respectively comprise first and second nucleic acid probes as described herein. In embodiments, the kit further comprises a reagent for conjugating the detection reagent to a detectable label or a nucleic acid probe. Conjugation of detection reagents to detectable labels and/or nucleic acid probes are further described, e.g., in WO 2020/180645.

In embodiments, the detection reagent is lyophilized. In embodiments, the detection reagent is provided in solution. In embodiments, the binding reagent is immobilized on the binding domain. In embodiments, the binding reagent is provided in solution. In embodiments, the reagents and other components of the kit are provided separately. In embodiments, the kit components are provided separately according to their optimal shipping or storage temperatures.

Reagents and methods for immobilizing binding reagents to surfaces, e.g., via targeting agents/targeting agent complements, linking agents/supplemental linking agents, and bridging agents are described herein. In embodiments, the surface is a plate. In embodiments, the surface is a multi-well plate. Non-limiting examples of plates include the MSD® SECTOR™ and MSD QUICKPLEX® assay plates, e.g., MSD® GOLD™ 96-well Small Spot Streptavidin plate. In embodiments, the surface is a particle. In some embodiments, the particle comprises a microsphere. In embodiments, the particle comprises a paramagnetic bead. In embodiments, the surface is a cartridge. In embodiments, the surface comprises an electrode. In embodiments, the electrode is a carbon ink electrode.

In embodiments, the kit further comprises a calibration reagent. In embodiments, the calibration reagent comprises a known quantity of the biomarker as described herein. In embodiments, multiple calibration reagents comprise a range of concentrations of the biomarker. In embodiments, the multiple calibration reagents comprise concentrations of the biomarker near the upper and lower limits of quantitation for the immunoassay. In embodiments, the multiple concentrations of the calibration reagent span the entire dynamic range of the immunoassay. In embodiments, the calibration reagent comprises an antibody biomarker. In embodiments, the antibody biomarker is a neutralizing antibody as described herein. In embodiments, the neutralizing antibody is a monoclonal antibody. In embodiments, the calibration reagent comprises a neutralizing antibody that specifically binds an MPXV and/or VACV antigen described herein. In embodiments, the calibration reagent is derived from human serum known to contain one or more antibodies that specifically bind to one or more viral antigens described herein. In embodiments, the one or more antibodies is human IgG, human IgM, or a combination thereof.

In embodiments, the kit further comprises a control reagent. In embodiments, the control reagent is a positive control reagent. In embodiments, the control reagent is a negative control reagent. In embodiments, the positive or negative control reagent is used to provide a basis of comparison for the biological sample to be tested with the immunoassays described herein. In embodiments, the positive control reagent comprises multiple concentrations of the biomarker. In embodiments, the positive control reagent comprises an antibody. In embodiments, the positive control reagent comprises human IgG, IgM, IgA, or a combination thereof. In embodiments, the negative control reagent comprises BSA.

In embodiments, the calibration and/or control reagent is lyophilized. In embodiments, the calibration and/or control reagent is provided in solution. In embodiments, the kit further comprises a diluent for preparing multiple concentrations of the calibration and/or control reagent. In embodiments, the kit comprises multiple calibration reagents at multiple concentrations, e.g., two or more, three or more, four or more, or five or more concentrations. In embodiments, the multiple concentrations of calibration reagents are used to calculate a standard curve. In embodiments, the multiple concentrations of calibration reagents provide thresholds indicating low, medium, or high levels of the biomarker being measured.

In embodiments, the kit further comprises a sample collection device. In embodiments, the sample collection device is an applicator stick. In embodiments, the sample collection device is a swab. In embodiments, the sample collection device is a tissue scraper. In embodiments, the sample collection device is a vial or container for collecting a liquid sample.

In embodiments, the kit further comprises one or more of a buffer, e.g., assay buffer, reconstitution buffer, storage buffer, read buffer, wash buffer and the like; a diluent; a blocking solution; an assay consumable, e.g., assay modules, vials, tubes, liquid handling and transfer devices such as pipette tips, covers and seals, racks, labels, and the like; an assay instrument; and/or instructions for carrying out the assay.

In embodiments, the kit comprises lyophilized reagents, e.g., binding reagent, detection reagent, calibration reagent, and/or control reagent. In embodiments, the kit comprises one or more solutions to reconstitute the lyophilized reagents.

In embodiments, a kit comprising the components above include stock concentrations of the components that are 5×, 10×, 20×, 30×, 40×, 50×, 60×, 70×, 80×, 90×, 100×, 125×, 150× or higher fold concentrations of the working concentrations of the immunoassays herein.

All references cited herein, including patents, patent applications, papers, textbooks and the like, and the references cited therein, to the extent that they are not already, are hereby incorporated herein by reference in their entirety.

### EXAMPLES

### Example 1. Serology Assays

Serology assays according to embodiments herein are performed on serum and/or plasma samples from subjects. Levels of IgG, IgM, and IgA antibodies are measured using multiplexes of viral antigens coated on plates using a two-step immunoassay. The multiplex of viral proteins is shown in Table 3.

TABLE 3
Monkeypox and Vaccinia Virus Antigens

| Monkeypox | Vaccinia |
|---|---|
| M1R | L1R |
| A29L | A27L |
| A35R | A33R |
| B6R | B5R |
| E8L | D8L |
| A30L | A28L |

The plates are 96-well plates, each well containing ten binding domains (“spots”) as shown in FIG. 2B. Each viral antigen is coated onto the plate in its assigned spot. Exemplary spot patterns are shown in Tables 4 and 5.

TABLE 4
Exemplary Spot Pattern-Panel 1

| Spot No. | Antigen |
|---|---|
| Spot 1 | VACV A27L |
| Spot 2 | VACV A33R |
| Spot 3 | VACV B5R |
| Spot 4 | VACV D8L |
| Spot 5 | BSA |
| Spot 6 | BSA |
| Spot 7 | MPXV E8L |
| Spot 8 | MPXV B6R |
| Spot 9 | MPXV A35R |
| Spot 10 | MPXV A29L |

TABLE 5
Exemplary Spot Pattern-Panel 2

| Spot No. | Antigen |
|---|---|
| Spot 1 | VACV A28 |
| Spot 2 | BSA |
| Spot 3 | VACV L1R |
| Spot 4 | BSA |
| Spot 5 | BSA |
| Spot 6 | BSA |
| Spot 7 | BSA |
| Spot 8 | MPXV M1R |
| Spot 9 | BSA |
| Spot 10 | MPXV A30L |

TABLE 6
Exemplary Spot Pattern-Panel 3

| Spot No. | Antigen |
|---|---|
| Spot 1 | VACV A27L |
| Spot 2 | VACV A33R |
| Spot 3 | VACV B5R |
| Spot 4 | VACV D8L |
| Spot 5 | VACV L1R |
| Spot 6 | MPXV M1R |
| Spot 7 | MPXV E8L |
| Spot 8 | MPXV B6R |
| Spot 9 | MPXV A35R |
| Spot 10 | MPXV A29L |

Samples (25 μL/well) are added to assay diluent (25 μL/well) preloaded into the well. Each sample is loaded in duplicate on three plates designated for measurements of IgG, IgM, and IgA antibodies (total volume requirement for saliva=300 μL). After incubation for about 1 hour to allow antibodies to bind the immobilized antigens, wells are aspirated to remove sample and washed three times.

In a classical serology assay format, a detection antibody specific for human IgG, IgM, or IgA is added to the corresponding wells. In a bridging serology assay format, a detection antigen, i.e., a second copy of the viral antigens described above, is added to the corresponding wells. The detection antibody or detection antigen is labeled with an electrochemiluminescence (ECL) probe. After incubation for 1 hour, wells are aspirated to remove unbound secondary antibody and washed three times. Read buffer is added, and the plates are read.

### Example 2. Serology Assay Protocol

An exemplary protocol for performing a serology assay of Example 1 includes:

Step 1. Prepare Plate.

The plate contains, e.g., the spot patterns according to any of Tables 4, 5, and 6. Plate preparation can include: (i) removing the plate from its packaging; (ii) adding a blocker solution, to the plate, e.g., at about 150 μL/well of MSD® Blocker A; (iii) sealing the plate with an adhesive plate seal; and (iv) incubating the plate, e.g., at room temperature with shaking (˜700 rpm) for at least 30 minutes.

During the plate incubation, the calibrators, controls, and samples may be prepared.

Calibrator Preparation. Prepare dilutions of a concentrated calibrator solution for establishing a calibrator curve, e.g., a 7-point calibration curve.

Control Preparation. Two positive controls and one negative control may be used. The positive controls may have assigned concentrations of an immunoglobin, e.g., human IgG, against the antigens in Panels 1, 2, and/or 3. The negative control does not include the immunoglobulin.

Sample Preparation. Samples may be diluted, e.g., with a diluent, about 100-fold to about 500-fold.

Step 2. Calibrators, Controls, and Sample Addition.

Following blocker solution incubation step, wash the plate at least 3 times, e.g., with at least 150 μL/well of a wash buffer. Then, add the samples, calibrators, and controls prepared above to the plate, e.g., at 50 μL/well. Seal the plate with an adhesive plate seal and incubate, e.g., at room temperature with shaking (˜700 rpm) for 2 hours.

During the plate incubation, the detection antibody solution may be prepared.

Detection Antibody Solution Preparation. The detection antibody may be supplied as a stock solution, e.g., a 200× stock solution, and should be diluted to a 1× working solution.

Step 3. Detection Antibody Addition.

Following the sample incubation step, wash the plate at least 3 times, e.g., with at least 150 μL/well of a wash buffer. Then, add the 1× detection antibody solution prepared above to the plate, e.g., at 50 μL/well. Seal the plate with an adhesive plate seal and incubate, e.g., at room temperature with shaking (˜700 rpm) for 1 hour.

Step 4. Read Buffer Addition.

Following the detection antibody incubation step, wash the plate at least 3 times, e.g., with at least 150 μL/well of a wash buffer. Then, add a read buffer, e.g., MSD GOLD® Read Buffer to the plate, e.g., at 150 μL/well. Read the plate, e.g., on an MSD instrument, immediately after adding the read buffer, without incubation or shaking.

1. A kit for detecting one or more antibody biomarkers of interest in a sample, the kit comprising, in one or more vials, containers, or compartments:
a. a surface comprising a panel of antigens immobilized on one or more binding domains, wherein each binding domain comprises an antigen of the panel of antigens immobilized thereon, wherein the panel of antigens comprises a monkeypox virus (MPXV) protein, a vaccinia virus (VACV) protein, or combination thereof; and
b. one or more detection reagents, wherein each detection reagent comprises a detection antibody, a detection antigen, or a detection competitor.

2. The kit of claim 1, wherein the MPXV protein comprises MIR, A29L, A35R, B6R, E8L, A30L, or combination thereof, and wherein the VACV protein comprises L1R, A27L, A33R, B5R, D8L, A28L, or combination thereof.

3. The kit of claim 1, wherein the panel of antigens comprises (i) the MPXV proteins MIR, E8L, B6R, A35R, and A29L; and (ii) the VACV proteins A27L, A33R, B5R, D8L, and L1R.

4. The kit of claim 1, wherein the antibody biomarker is IgG, IgM, IgA, or an antigen-binding fragment thereof.

5. The kit of claim 1, wherein the detection reagent comprises a detection antibody, wherein the detection antibody comprises an anti-IgG antibody, anti-IgM antibody, anti-IgA antibody, or combination thereof.

6. The kit of claim 1, wherein each detection reagent comprises a detectable label; or
wherein the kit further comprises a reagent for conjugating a detection label to each detection reagent.

7. (canceled)

8. The kit of claim 6, wherein the detectable label is capable of being detected by light scattering, optical absorbance, fluorescence, chemiluminescence, electrochemiluminescence (ECL), bioluminescence, phosphorescence, radioactivity, magnetic field, or combination thereof.

9. The kit of claim 1, wherein the surface comprises an electrode.

10. The kit of claim 1, wherein the surface comprises a well of a multi-well plate, and wherein each well comprises one to ten binding domains; or wherein the surface comprises a particle comprising one or more binding domains, and wherein the kit comprises one or more particles.

11. (canceled)

12. The kit of claim 1, further comprising a calibration reagent, a buffer, a diluent, a sample collection device, or combination thereof.

13. A method of detecting one or more antibody biomarkers of interest in a sample, comprising:
(a) forming one or more binding complexes, wherein each binding complex comprises an antigen; an antibody biomarker that binds to the antigen; and a detection reagent, wherein the antigen comprises a monkeypox virus (MPXV) protein, a vaccinia virus (VACV) protein, or combination thereof; and
(b) detecting the one or more binding complexes, thereby detecting the one or more antibody biomarkers in the sample.

14. The method of claim 13, wherein the antigen of each binding complex is immobilized to a distinct binding domain on a surface prior to or during the forming of (a); or
wherein the method further comprises immobilizing the antigen of each binding complex to a distinct binding domain on a surface following formation of the one or more binding complexes.

15. (canceled)

16. The method of claim 13, wherein step (a) comprises:
contacting the sample comprising the antibody biomarker with the antigen and the detection reagent simultaneously or substantially simultaneously;
contacting the sample comprising the antibody biomarker with: (i) first, the antigen; and (ii) second, the detection reagent;
or
contacting the sample comprising the antibody biomarker with: (i) first, the detection reagent; and (ii) second, the antigen.

17. (canceled)

18. (canceled)

19. A method of detecting one or more antibody biomarkers of interest in a sample, comprising:
(a) contacting the sample with a surface comprising a panel of antigens immobilized on one or more binding domains, wherein each binding domain comprises an antigen of the panel of antigens immobilized thereon, wherein the panel of antigens comprises a monkeypox virus (MPXV) protein, a vaccinia virus (VACV) protein, or combination thereof;
(b) forming a binding complex in each binding domain, wherein each binding complex comprises the antigen and an antibody biomarker that binds to the antigen;
(c) contacting the binding complex in each binding domain with a detection reagent; and
(d) detecting the binding complexes on the surface, thereby detecting the one or more antibody biomarkers in the sample.

20. The method of claim 13, wherein the MPXV protein comprises MIR, A29L, A35R, B6R, E8L, A30L, or combination thereof, and wherein the VACV protein comprises L1R, A27L, A33R, B5R, D8L, A28L, or combination thereof.

21. The method of claim 19, wherein the panel of antigens comprises (i) the MPXV proteins MIR, E8L, B6R, A35R, and A29L; and (ii) the VACV proteins A27L, A33R, B5R, D8L, and L1R.

22. The method of claim 13, wherein;
(i) the antibody biomarker is IgG, IgM, IgA, or an antigen-binding fragment thereof;
(ii) the detection reagent comprises a detection antibody, wherein the detection antibody comprises an anti-IgG antibody, anti-IgM antibody, anti-IgA antibody, or combination thereof; or wherein the detection reagent comprises a detection antigen or a detection competitor;
(iii) the detection reagent comprises a detectable label, wherein the detectable label is capable of being detected by light scattering, optical absorbance, fluorescence, chemiluminescence, electrochemiluminescence (ECL), bioluminescence, phosphorescence, radioactivity, magnetic field, or combination thereof;
or
(iv) any combination of (i) to (iii).

23. (canceled)

24. (canceled)

25. (canceled)

26. (canceled)

27. The method claim 14, wherein the surface comprises an electrode, and wherein:
the surface comprises a well of a multi-well plate, and wherein each well comprises one to ten binding domains; or
the surface comprises a particle comprising one or more binding domains, and wherein the contacting comprises contacting the sample with one or more particles.

28. (canceled)

29. (canceled)

30. The method of claim 14, wherein the detection reagent comprises an ECL label, the surface comprises an electrode, and the detecting comprises applying a voltage to the surface and measuring an ECL signal generated from the ECL label on the detection reagent.

31. The method of claim 13, wherein the sample comprises whole blood, serum, plasma, cerebrospinal fluid, urine, saliva, endotracheal aspirate, nasal swab, a wastewater sample, or an extraction or purification therefrom, or dilution thereof.

32. (canceled)
